(19) United States
(12) Patent Application Publication
(10) Pub. No.: US 2024/0016916 A1
(43) Pub. Date: Jan. 18, 2024

(54) BACTERIAL OUTER MEMBRANE VESICLES CARRYING CORONAVIRUS POLYPEPTIDES, METHOD OF PREPARATION, COMPOSITIONS AND USE THEREOF

(71) Applicant: BIOMVIS S.R.L., Siena (SI) (IT)

(72) Inventors: Alberto Grandi, SIENA (IT); Guido Grandi, SEGRATE (MI) (IT); Massimo Pizzato, TRENTO (IT); Michele Tomasi, TRENTO (IT)

(73) Assignee: BIOMVIS S.R.L., Siena (SI) (IT)

(21) Appl. No.: 17/998,702

(22) PCT Filed: May 14, 2021
(86) PCT No.: PCT/EP2021/062875

§ 371 Date: Nov. 14, 2022

(30) Foreign Application Priority Data
May 15, 2020 (EP) 20175072.6

(51) Int. Cl. A61K 39/215; C12N 1/20; C07K 14/005; A61P 31/14

(52) U.S. Cl. CPC A61K 39/215; C12N 1/20; A61K 2039/6018; A61P 31/14; C12N 2770/20022; C07K 14/005

(57) ABSTRACT

This invention relates to genetically modified gram-negative bacteria specifically designed to release engineered Outer Membrane Vesicles (OMVs) carrying Coronavirus (poly)peptides. The invention further provides methods for the preparation of the OMVs, immunogenic compositions containing them and the use thereof as vaccines for the prophylaxis and treatment of SARS-CoV2 infections.

This application is a U.S. national stage of PCT/EP2021/062875 filed on 14 May 2021, which claims priority to and the benefit European Patent Application No. 20175072.6 filed on 15 May 2020, the contents of which are all incorporated herein by reference in their entireties.

Sequence listing Sequence.txt created Nov. 11, 2022 and of size of 34,859 bytes is incorporated herein by reference.

This invention relates to genetically modified gram-negative bacteria specifically designed to release engineered Outer Membrane Vesicles (OMVs) carrying Coronavirus (poly)peptides. The invention further provides methods for the preparation of the OMVs, immunogenic compositions containing them and the use thereof as vaccines for the prophylaxis and treatment of SARS-CoV2 infections.

### BACKGROUND ART

### Coronavirus Infections and Epidemics

The world has witnessed and documented the progress of several epidemics in the last one hundred years, including outbreaks of new influenza viruses, vector-borne flaviviruses such as West Nile and Zika viruses, HIV and coronaviruses. Some of these, such as SARS-CoV-2, the recent cause of COVID-19, went on to originate pandemic infections which brought social and economic catastrophic consequences worldwide. Given the zoonotic source of novel human viruses, it is impossible to predict the time and the location of their appearance into the human population. Likewise, it is often impossible to detect human infections with new viruses until it is too late to prevent their spreading on a global scale.

In the last 20 years, there have been three outbreaks of novel coronaviruses, SARS-CoV, MERS and SARS-CoV-2. With at least 200 species documented in different animals, coronaviruses will undoubtedly represent a constant threat in the future, making the identification of an effective strategy to deal with sudden outbreaks a pressing priority.

Similarly to SARS-CoV and MERS, SARS-CoV-2 causes acute infections by replicating in the respiratory epithelium and damaging the lung in a very short period of only a few days. As for many acute infections, viruses spread rapidly across tissues and cause severe damage before a full adaptive immune response can come into force to clear the infection. As well documented with SARS, a proinflammatory component also contributes to lung injury (He et al., (2006) J. Pathol. 210, 288-297; Ward et al., (2005) Antivir. Ther. 10, 263-275) crucially aggravating the clinical outcome of the most severe cases. However, while the lethality varies between 3% for SARS-CoV-2 and 32% for MERS, in the majority of cases, patients fully recover in a four-week average period after the initial infection. Given the genetic similarity between SARS-CoV and SARS-CoV-2 (which share 74.5% identity at amino acid level and use the same cellular receptor ACE2), and the analogy of the pathology they cause, the information acquired with SARS-CoV during the last 20 years provides valuable knowledge applied to SARS-CoC-2. SARS-CoV is known to induce strong immunological responses which generate abundant IgM and IgG in 100% of convalescent patients (Li et al., (2003) N. Engl. J. Med. 349, 508-509). These antibodies target primarily the nucleocapsid N protein and the spike S protein, the latter providing the strong neutralizing capacity observed in patients sera (Zhang, L et al. (2006) J. Med. Virol 78, 1-8). Importantly, both the level and the persistence of neutralizing antibodies (NAb) correlate with patient recovery, underscoring the crucial role played by NAb in determining the ultimate disease outcome.

### Therapeutic and Prophylactic Strategies Against SARS-CoV2

On the basis of what said above, the capabilities to implement timely prophylactic and therapeutic strategies is paramount. The two main possible approaches are 1) passive immunization with neutralizing monoclonal antibodies (Nab), and 2) vaccination.

Similarly to SARS-CoV, SARS-CoV-2 infects cells by binding to the ACE2 protein on the cell membrane, with a well-defined 190 amino acids receptor-binding domain (RBD) within the virus spike protein. Receptor binding is the key event required for the virus in order to fuse with the cell membrane and to access the cell cytoplasm. Accordingly, a significant fraction of the immunoglobulins found in convalescent SARS patients contain neutralizing antibodies (Zhang, L et al. (2006) J. Med. Virol 78, 1-8), the large majority of which target the RBD. From this evidence, monoclonal antibodies against the RBD would benefit the clinical outcome. However, caution has to be exerted before considering a clinical application of nAbs for COVID-19. There has been strong evidence indicating that neutralizing antibodies against SARS-CoV contribute crucially to the exacerbation of an inflammation that is a driving factor towards fatality. In SARS-CoV macaque models, adoptive transfer of anti-S-IgG nAb, led to severe lung injury, coupled to a reduction of wound healing-polarized macrophages (Liu, L, et al. (2019) JCI Insight 4). This correlates with a previous observation that patients who develop a high amount of neutralizing antibodies too soon after infection have a significantly higher chance of dying from the disease (Zhang, L et al. (2006) J. Med. Virol 78, 1-8). How could antibodies contribute to disease exacerbation? Two non-mutually exclusive explanations contemplate the involvement of the interaction of the antibody Fc portion with FcR on immune cells: i) anti-spike antibodies drive excessive production of inflammatory cytokines by favoring the accumulation of pro-inflammatory macrophages via interaction of Fc to FcR (Liu, L, et al. (2019) JCI Insight 4) and ii) antibody-dependent enhancement of infectivity, documented by different groups (Jaume, M et al., (2011) J. Virol. 85, 10582-10597; Wang, S F et al., (2014) Biochem. Biophys. Res. Commun. 451, 208-214; Yang, Z. et al. (2005) Proc. Natl. Acad. Sci. 102, 797-801; Yip, M S et al., (2014) Virol. J. 11, 82) and mediated by anti-spike antibodies, contributes to persistent virus replication and a resulting exacerbation of inflammation.

In conclusion, the quality and quantity of antibodies to be administered to the patients appear to be crucial for an effective therapy. Theoretically, antibodies should strongly bind the region of the viral Spike protein involved in the interaction with the ACE2 receptor, while sparing regions outside the receptor binding domain. Moreover, considering the variability of the Spike protein among different SARS-CoV2 isolates, antibodies targeting conserved regions should be selected.

Vaccination is undoubtedly the most effective and desirable prophylactic tool (Enjuanes L et al. (2016) Advances in Virus Research, Coronaviruses. Academic Press, pp. 245-286).

Previous studies on SARS have shown that vaccines targeting S protein are effective in inducing potent systemic immune responses with neutralizing activities that completely protects vaccinated animals from virus challenge. Moreover, recent studies have shown that a recombinant vaccinia virus Ankara (MVA) expressing full length S protein of MERS-CoV produced high levels of antibodies that neutralize MERS-CoV infection (Song F et al., (2013) J. Virol. 87,11950-11954). Finally, it has been shown that intranasal vaccination with recombinant Adeno-Associated Virus encoding the receptor-binding domain (RBD) elicited strong antibody and T cell responses both systemically and in the lung (Du L. et al. (2008) J. Immunol. 180, 948-956). T cell responses appear to be particularly important for a full blown protective immunity and T cell epitopes are not restricted to the S protein. Long lasting protection against SARS in intranasally-immunized mice was shown to be mediated by the elicitation of alveolar memory CD4+ T recognizing a conserved CD4+ T cell epitope of the necleocapsid N protein of SARS (Zhao et al. (2016), Immunity, 44, 1379).

Based on the SARS and MERS-CoV experience, several vaccines are currently under evaluation for SARS-CoV2, and some of them have already reached the Phase I in humans. In a recent article in Science (Jon Cohen (2020) Science, 368, 14-16) it has been reported that as of Apr. 3, 2020, 52 vaccine candidates are under development and other are expected to emerge soon. Such vaccine candidates are based on different platforms including protein subunit vaccines formulated with different adjuvants, DNA and RNA-based vaccines, non-replicating and replicating vectors, inactivated (killed virus), attenuated virus and virus-like particles. In general, all these vaccines aim at inducing antibodies targeting the viral S protein, thus preventing the interaction of the virus with its receptor.

While the elicitation of anti-S protein antibodies is surely a strong correlate of protective immunity, the absence of selectivity in epitope recognition could legitimate concerns similar to those highlighted for passive immunization. Indeed, certain vaccination strategies can aggravate the pulmonary injury following infection of animal models (Bolles, M et al., (2011); J. Virol. 85, 12201-12215. Clay, C. et al. (2012) J. Virol. 86, 4234-4244; Tseng, C T et al. (2012) PLOS ONE 7, e35421). This might also be due to the elicitation of a Th2-skewed immune response, which could enhance the inflammatory reaction occurring in the lungs during viral infection.

Therefore, vaccines capable of eliciting a selective profile of neutralizing antibodies and T cells in a Th1-skewed environment should be particularly beneficial.

### Bacterial Outer Membrane Vesicles (OMVs)

All Gram-negative bacteria spontaneously release outer membrane vesicles (OMVs) during growth both in vitro and in vivo. OMVs are closed spheroid particles, 20-300 nm in diameter, generated through a “budding out” of the bacterial outer membrane. Consistent with that, the majority of OMV components are represented by LPS, glycerophospholipids, outer membrane proteins, lipoproteins and periplasmic proteins (A. Kulp and Kuehn M. J. (2010) Annu. Rev. Microbiol. 64, 163-184; T. N. Ellis and Kuehn M. J. (2010) Microbiol. Mol. Biol. Rev. 74, 81-94).

OMVs represent a distinct secretory pathway with a multitude of functions, including inter and intra species cell-to-cell cross-talk, biofilm formation, genetic transformation, defense against host immune responses and toxin and virulence factor delivery to host cells (A. Kulp and Kuehn M. J. (2010) Annu. Rev. Microbiol. 64, 163-184). OMVs interaction to host cells can occur by endocytosis after binding to host cell receptors or lipid rafts. Alternatively, OMVs have been reported to fuse to host cell membrane, leading to the direct release of their content into the cytoplasm of the host cells (A. Kulp and Kuehn M. J. (2010) Annu. Rev. Microbiol. 64, 163-184; T. N. Ellis and Kuehen M. J. (2010) Micrbiol. Mol. Biol. Rev. 74, 81-94).

### OMVs as Vaccines

OMVs purified from several pathogens, including Neisseria, Salmonella, Pseudomonas, Vibrio cholerae Burkholderia, and E. coli, induce potent protective immune responses against the pathogens they derive from (B. S. Collins (2011) Discovery Medicine, 12 7-15), and highly efficacious anti-Neisseria OMV-based vaccines are already available for human use and others are in advanced clinical phases (S. N. Ladhani et al. Arch. Dis. Child. 101, 91-95 (2016); D. Serruto, et al. Vaccine. 30, B87—B97 (2012); C. Gerke et al., PLoS One. 10, e0134478 (2015); O. Rossi et al., Clin. Vaccine Immunol. 23, 304-314 (2016).

Such remarkable protection is attributed to two main properties of OMVs. First, they carry the proper immunogenic and protective antigens, which, in extracellular pathogens, usually reside on the surface and therefore are naturally incorporated in OMVs. Indeed, OMV immunization induces potent antibody responses against the major membrane-associated antigens. However, OMV immunogenicity is not restricted to antibody responses. For instance, mice immunized with Salmonella OMVs develop robust Salmonella-specific B and T cell responses, and OMVs stimulate IFN-γ production by a large proportion of CD4+ T cells from mice previously infected with Salmonella, indicating that OMVs are an abundant source of antigens recognized by Salmonella-specific CD4+ T cells (R. C. Alaniz et al., (2007) J. Immunol. 179, 7692-7701). Second, OMVs possess a strong “built-in” adjuvanticity since they carry many of the bacterial Pathogen-Associated-Molecular Patterns (PAMPs) which, by binding to pathogen recognition receptors (PRRs), play a key role in stimulating innate immunity and in promoting adaptive immune responses. OMV-associated PAMPs include LPS which, in concert with MD-2 and CD14, binds TLR-4, lipoproteins whose acylpeptide derivatives interact with TLR-1/2 and 2/6 heterodimers, and peptidoglycan whose degradation products bind to intracellular NOD1/2 (A. Moshiri et al., Hum. Vaccines. Immunother (2012) 8, 953-955; T. N. Ellis et al., (2010) Inn. Immun. 78, 3822-3831; M. Kaparakis et al., (2010) Cell. Miocrobiol. 12, 372-385). The engagement of this group of PRRs results in the activation of transcription factors (NF-kB) and the consequent expression of specific cytokines. Interestingly, LPS, lipoproteins and peptidoglycan can work synergistically, thus potentiating the built-in adjuvanticity of OMVs (D. J. Chen et al., (2010) PNAS, 107, 3099-3104).

OMVs also have the capacity to induce protection at the mucosal level. Protection at the mucosal sites is known to be at least partially mediated by the presence of pathogen-specific IgAs and Th17 cells. In particular, a growing body of evidence suggests that Th17 cells have evolved to mediate protective immunity against a variety of pathogens at different mucosal sites. Interestingly, Th17 cells have recently also been shown to play a crucial role in the generation of vaccine-induced protective responses. For instance, it has been reported that in mice whole cell pertussis vaccines (Pw) induce Th17 cells and neutralization of IL-17 after vaccination reduces protection against a pulmonary challenge with B. pertussis. Similarly, in a CD4+ T cell dependent, antibody-independent model of vaccine-induced protection following S. pneumoniae challenge, treatment with anti-IL-17 antibodies resulted in reduced immunity to pneumococcal colonization compared to the control serum treated mice (Malley R, et al. (2006) Infect Immun., 74:2187-95). Elicitation of IgAs and Th17 cells by OMVs has been well documented and this can explain mechanistically the good protective activities of OMVs against several mucosal pathogens. For instance, immunization with Vibrio cholerae-derived OMVs protects rabbits against Vibrio cholerae oral challenge (Roy N. et al. (2010) Immunol. Clinical Microbiol. 60, 18-27) and Pasteurella multocida-derived and Mannheimia haemolytica-derived OMVs protect mice from oral challenge with P. multocida (Roier S. et al., (2013) Int. J. Med. Microbiol. 303, 247-256). In addition, intranasal immunization with Porphyromonas gingivalis OMVs elicits potent IgA production at both serum and mucosal level and immunization with Escherichia coli-derived OMVs prevent bacteria-induced lethality. Protective effect of Escherichia coli-derived OMVs is primarily mediated by OMV-specific, IFN-γ and IL-17 producing, T cells (Kim O Y et al., (2013) J. Immunol. 190, 4092-4102).

Additional advantages of OMVs as vaccine platform include:
1. OMVs are amenable for large scale production—In general, the amount of OMVs released by Gram-negative bacteria when grown under laboratory conditions is too low to allow their exploitation in biotechnological applications. However, two approaches can be used to enhance the yields of OMVs and make them compatible with industrial applications. The first one exploits the addition of mild detergents to the bacterial biomass to promote the vesiculation process and, at the same time, to decrease the level of OMV reactogenicity by removing a substantial amount of LPS (Fredriksen J. H. et al, (1991) NIPH Ann. 14, 67-79). Although this process has been proved to produce safe and effective vaccines against Meningococcal B (Granoff D. (2010), Clin. Infect. Dis. 50, S54-S65; Crum-Cianflone N, Sullivan E. (2016) Meningococcal vaccinations. Infect Dis Ther., 5, 89-112) its main drawback is that the detergent treatment favors bacterial cell lysis with the consequence that the OMV preparations are heavily contaminated with cytoplasmic proteins (Ferrari et al., (2006) Proteomics, 6, 1856-1866). The second approach to enhance OMV production is to insert into the genome of the OMV-producing strain mutations that enhance vesiculation. For instance, in Neisseria meningitidis, a mutation in the gna33 gene, encoding a glucosyltransferase, has been shown to drive the release of several milligrams of vesicles per liter in the culture supernatant (Ferrari et al., (2006) Proteomics, 6, 1856-1866).

Similar quantities of vesicles are obtained from Escherichia coli strains carrying deletions in the genes encoding the Tol/Pal system (a protein complex involved in the connection of the inner membrane with the outer membrane) (Bernadac A. et al., (1998) J. Bacteriol. 180, 4872-4878) and in the ompA gene, encoding one of the major outer membrane proteins of E. coli (Fantappie et al., (2014) Journal of Extracellular Vesicles, 3, 24015). Deletion of the VacJ/Yrb ABC (ATP-binding cassette) transport system, a proposed phospholipid transporter, was also shown to increase OMVs production in two distantly related Gram-negative bacteria, Haemophilus influenzae and Vibrio cholerae (Roier S. et al, (2016) Nat. Commun. 7, 10515). Such quantities make the production process of OMVs highly efficient and inexpensive. A number of other mutations have been described that enhance the production of OMVs in several Gram negative bacteria, including Salmonella and E. coli (Deatherage B. L. et al. (2009) Mol. Microbiol. 72, 1395-1407; McBroom A. J. and Kuehen M. J. (2007) Mol. Microbiol. 63, 545-558). Furthermore, a high-throughput method developed to measure vesiculation values for the whole genome knock out library of E. coli mutant strains (Keio collection (Baba T. et al. (2006) Molecular System Biology DOI: 10.1038/msb4100050)) revealed 171 mutant strains with significant vesiculation phenotypes. Of these, 73 exhibited over-vesiculation phenotypes and 98 showed under-vesiculation phenotypes (Kulp A. J. et al (2015) PLos ONE 10(9): e0139200). Finally, it has been recently demonstrated that E. coli strains carrying multiple mutations of genes encoding periplasmic and membrane associated proteins feature a particularly high hypervesiculating phenotype (PCT/EP2020/060762).

As far as the purification of OMVs from the culture supernatant is concerned, centrifugation and tangential flow filtration (TFF) are commonly used. The yield of OMV production using centrifugation couple to TFF can easily exceed 100 mg/liter of culture (Berlanda Scorza F. et al., (2012) PlosOne 7, e35616) and therefore the process is perfectly compatible with large scale production.
2. OMVs can be manipulated in their protein content by genetic engineering. This feature was demonstrated for the first time by Kesty and Kuehn who showed that Yersinia enterocolitica outer membrane protein Ail assembled on OMVs surface when expressed in E. coli, and that the GFP fluorescence protein fused to the “twin arginine transport (Tat)” signal sequence was incorporated in the OMV lumen (N. C. Kesty and Kuhen M. J. (2004) J. Biol. Chem. 279, 2069-2076). Following the observation by Kesty and Kuehn, an increasing number of heterologous proteins have been successfully delivered to OMVs using a variety of strategies. For instance, heterologous antigens have been delivered to the surface of OMVs by fusing them to the β-barrel forming autotransporter ADA and to hemolysin ClyA, two proteins that naturally compartmentalized into E. coli OMVs (J. Schroeder and Aebischer T. (2009) Vaccine, 27, 6748-6754; D. J. Chen et al., (2010) PNAS, 107, 3099-3104). Recently, heterologous antigens from Group A Streptococcus and Group B Streptococcus were delivered to the lumen of E. coli vesicles by fusing their coding sequences to the leader peptide of E. coli OmpA. Interestingly, when the recombinant vesicles were used to immunize mice, they elicited high titers of functional antibodies against the heterologous antigens, despite their luminal location (Fantappiè et al., (2014) Journal of Extracellular Vesicles, 3, 24015). More recently, we have shown that heterologous antigens can be delivered to the vesicular compartment by expressing them as lipoproteins in the OMV-producing strain (WO2015/144691, WO2006/024954, Fantappie' et. al (2017) Mol. Cell. Proteomics 16:1348-1364; Irene et al (2019) PNAS 116, 21780-21788). Interestingly, lipoproteins can also serve as chaperones to deliver foreign polypeptides to the OMVs compartment, thus allowing the decoration of vesicles with a variety of polypeptides and their exploitation in different biotechnological applications, including vaccines and immunotherapy. Finally, we have recently shown that heterologous proteins and polypeptides accumulate at high levels in the vesicular compartment of a novel E. coli derivative carrying the inactivation of 60 genes encoding endogenous OMV-associated proteins. Such engineered, proteome-minimized OMVs elicit potent immune responses against the heterologous antigens in experimental animals (PCT/EP2020/060762).

### DISCLOSURE OF THE INVENTION

The present invention is based on the finding that gram-negative bacteria can be engineered to release OMVs carrying SARS-CoV2 proteins fragments thereof or multiple fragment fusion products, and that the OMVs thereby produced are able to elicit neutralizing antibodies and protective T cells, making their use for vaccine purposes particularly convenient. The design of OMVs carrying specific SARS-CoV2 antigens or epitopes is particularly important not only to optimize the elicitation of protective immune responses but also to reduce the risk of production of non-neutralizing antibodies that could contribute to inflammation mediated by the engagement of macrophage Fc receptor.

According to a first embodiment, the invention provides a method of producing bacterial outer membrane vesicles (OMVs) carrying a SARS-CoV2 protein, a fragment thereof or a fusion of multiple SARS-CoV2 protein fragments, in the OMV lumen or membrane, said method comprising:
(i) culturing a Gram-negative bacterium expressing the SARS-CoV2 protein, a fragment thereof or a fusion of multiple fragments in the OMVs, in conditions suitable for vesiculation;
(ii) separating the OMVs from the bacterial culture,
and optionally,
(iii) purifying the OMVs.

The term “fragment” denotes an immunogenic portion, region or domain of a SARS-CoV2 protein, e.g. an epitope or an antigenic region, which is able to stimulate the production of antibodies against the SARS-CoV2 protein. Multiple copies of a SARS-CoV2 fragment or multiple fragments from the same or different SARS-CoV2 proteins can be fused to one another, optionally with interposition of a spacer, to generate a multiple fragment fusion product according to the invention. The spacer consists of a sequence of 2 to 10 amino acids, particularly from 2 to 6 and from 2 to 4 amino acids. Preferably the spacer sequence contains neutral amino acid residues, which are preferably selected from Gly and Ser.

The gram-negative bacterium to be engineered with the SARS-CoV2 protein, fragments or multiple fragment fusion products can be of the genus Escherichia, Pseudomonas, Neisseria or Shigella. E. coli strains are preferably used.

Preferably, in order to increase the amount of OMVs the bacterium is genetically modified by introducing gene-inactivating mutations in the ompA gene and in one or more of the following genes which encode proteins naturally present in the OMVs:
ybis, ais, eco, glpQ, mltA, proX, ydcL, glnH, efeO, bglX, agp, ygdI, yncD, slp, artI, yiaD, ompX, borD, yhiJ, emtA, fecA, nmpC, fhuA, hist, lamB, malE, malM, ygiW, cirA, fepA, loiP, yjeI, ecnB, rcsF, phoE, oppA, fkpA, ybaY, tsx, yggE, osmE, ygdR, yceI, bhsA, nlpE, pldA, yghJ, ydeN, ushA, mdoD, treA, bcsC, ftsP, ptrA, fadL, artJ, mlaA.

The further inactivation of the two genes msbB and pagP involved in the LPS biosynthetic pathway is particularly preferred as it enables the mutated strain to release significantly less reactogenic vesicles.

In a preferred embodiment of the invention the strain used for the production of the OMVs is an E. coli strain carrying all the above mentioned mutations (named BL21Δ60 strain).

The OMVs deprived of the proteins encoded by the genes identified above elicit higher protein-specific antibody titers and show reduced reactogenicity. Methods for the preparation of the mutant bacterial strains are disclosed in the co-pending application PCT/EP2020/060762 (WO2020/212524), in the applicant's name, the content of which is herein incorporated by reference. In particular, the methods and techniques for gene inactivation are disclosed at pages 10 and 11 and in the section “detailed description of invention” of the cross-referenced application, and specifically at pages 19 through 31 (“Selection of proteins to be eliminated from the OMVs” and “Inactivation of selected OMV proteins”).

The bacterium is cultured in conditions suitable for growth and vesiculation, which include the use of rich media such as LB supplemented with additional carbon and nitrogen sources, or chemically defined media using different carbohydrates as carbon sources. Growth temperatures typically vary from 20° C. to 37° C. and the supernatants containing the vesicles can be collected toward the end of the exponential phase or in the stationary phase of growth, depending upon the growth conditions in use. The conditions suitable for bacterial growth and vesiculation are known to anyone skilled in the art and are described for instance in Berlanda Scorza, F. et al. “High yield production process for Shigella outer membrane particles”, PLoS One 7, e35616 (2012).

The strains of the invention are genetically engineered to express the SARS-CoV2 proteins, fragments or multiple fragment fusion products either in the OMV lumen or in the OMV membrane. Luminal expression can be promoted by fusing SARS-CoV2 proteins, fragments or multiple fragment fusion products to a leader sequence, such as OmpA leader sequence, which delivers proteins/polypeptides to the periplasmic space. Membrane expression can be obtained by fusing the SARS-CoV2 proteins, fragments or multiple fragment fusion products to a lipoprotein leader sequence, such as Lpp leader sequence, which promote the integration of lipidated heterologous SARS-CoV2 polypeptides into the OMV membrane. Methods for generating lipoproteins containing the heterologous polypeptides and their delivery in the OMVs are disclosed in EP3312192.

Furthermore, according to the invention, SARS-CoV2 proteins, fragments or multiple fragment fusion products can be expressed in OMVs as fusions to carrier proteins, which promote the transport of passenger polypeptides to the lumen or to the membrane of OMVs. Preferably, the SARS-CoV2 proteins, fragments or multiple fragment fusion products are fused to the C-terminus of the Staphylococcus aureus FhuD2 protein, as disclosed in WO2019/170837. With the method of invention, an OMV or a mixture of OMVs can be loaded with a plurality of different SARS-CoV2 proteins, fragments or multiple fragment fusion products.

The fusion product of multiple SARS-CoV2 protein fragments is obtained by the fusion of either multiple copies of the same SARS-CoV2 protein fragment or of different SARS-CoV2 protein fragments.

Preferably, the SARS-CoV2 protein is selected from the SARS-CoV2 Spike protein and the Nucleocapsid (N) protein.

In one embodiment of the invention, the SARS-CoV2 protein fragment expressed in the lumen or in the membrane of the OMVs is the SARS-CoV2 Spike polypeptide consisting of the sequence SEQ ID NO:1 (Fragment 1), or a derivative thereof having at least 75%, preferably at least 85% sequence identity to SEQ ID NO:1:

PNITNLXPFGEVFNATRFASVYAWNRKRISNXVADYSVLYNSASFSTFKX
YGVSPTKLNDLXFTNVYADSFVIRGDEVRQIAPGQTGKIADYNYKLPDDF
TGXVIAWNSNNLDSKVGGNYNYLYRLFRKSNLKPFERDISTEIYQAGSTP
CNGVEGFNCYFPLQSYGFQPTNGVGYQPYRVVVLSFELLHAPATVXGPKK
STNLVKNKXVNFNFNGLTGTGVLTESNKKFLPFQQFGRDIADTTDAVRDP
QTLE,

wherein “X” represents any of the 20 natural amino acids, preferably Cysteine, Alanine or Serine (Fragment 1, SEQ ID NO:1).

As used herein, the term “derivative” refers to immunogenic (poly)peptides which retain the ability to generate anti-SARS-CoV2 antibodies when incorporated in the lumen or in the membrane of bacterial OMVs.

In another embodiment, the SARS-CoV2 protein fragment expressed in the lumen or in the membrane of the OMVs is the SARS-CoV2 Spike polypeptide consisting of the sequence SEQ ID NO:2 (Fragment 2) or a derivative thereof having at least 75%, preferably at least 85% sequence identity to SEQ ID NO:2:

(Fragment 2, SEQ ID NO: 2)
SNNLDSKVGGNYNYLYRLFRKSNLKPFERDISTEIYQAGSTPCNGVEGFN
CYFPLQSYGFQPTNGVGYQPYR.

In another embodiment, the SARS-CoV2 protein fragment expressed in the lumen or in the membrane of the OMVs is the SARS-CoV2 Spike polypeptide consisting of the sequence SEQ ID NO:3 (Fragment 3) or a derivative thereof having at least 75%, preferably at least 85% sequence identity to SEQ ID NO:3:

(Fragment 3, SEQ ID NO: 3)
SNNLDSKVGGNYNYLYRLFRKSNLK.

In another embodiment, the SARS-CoV2 protein fragment expressed in the lumen or in the membrane of the OMVs is the SARS-CoV2 Spike polypeptide consisting of the sequence SEQ ID NO:4 (Fragment 4) or a derivative thereof having at least 75%, preferably at least 85% sequence identity to SEQ ID NO:4:

(Fragment 4, SEQ ID NO: 4)
DISTEIYQAGSTPCNGVEGFNCYFPLQSYGFQPTNGVGYQPYR.

In another embodiment, the SARS-CoV2 protein fragment expressed in the lumen or in the membrane of the OMVs is the SARS-CoV2 Spike polypeptide consisting of the sequence SEQ ID NO:5 (Fragment 5) or a derivative thereof having at least 75%, preferably at least 85% sequence identity to SEQ ID NO:5:

(Fragment 5, SEQ ID NO: 5)
DIPIGAGIXASYQTQTNSPRRARSVASQSIIAY,
wherein “X” represents any of the 20 natural amino
acids, preferably Cysteine, Alanine or Serine.

In another embodiment, the SARS-CoV2 protein fragment expressed in the lumen or in the membrane of the OMVs is the SARS-CoV2 Nucleocapsid (N) polypeptide consisting of the sequence SEQ ID NO:6 (Fragment 6) or a derivative thereof having at least 75%, preferably at least 85% sequence identity to SEQ ID NO:6:

(Fragment 6, SEQ ID NO: 6)
LLNKHIDAYKTFP.

In another embodiment, the SARS-CoV2 fragment fusion product expressed in the lumen or in the membrane of the OMVs is the fusion of Fragments 2, 5 and 6, consisting of the sequence SEQ ID NO:7 (Fragment 7) or a derivative thereof having at least 75%, preferably at least 85% sequence identity to SEQ ID NO:7:

(Fragment 7, SEQ ID NO:)
SNNLDSKVGGNYNYLYRLFRKSNLKPFERDISTEIYQAGSTPXNGVEGFN
XYFPLQSYGFQPTNGVGYQPYRGSDIPIGAGIXASYQTQTNSPRRARSVA
SQSIIAYGSLLNKHIDAYKTFP,
wherein “X” represents any of the 20 natural amino
acids, preferably Cysteine, Alanine or Serine.

In another embodiment, the SARS-CoV2 fragment fusion product expressed in the lumen or in the membrane of the OMVs is the fusion of Fragments 4, 5 and 6, consisting of the sequence SEQ ID NO:8 (Fragment 8) or a derivative thereof having at least 75%, preferably at least 85% sequence identity to SEQ ID NO:8:

(Fragment 8, SEQ ID NO: 8)
DISTEIYQAGSTPXNGVEGFNXYFPLQSYGFQPTNGVGYQPYRGSDIPIG
AGIXASYQTQTNSPRRARSVASQSIIAYGSLLNKHIDAYKTFP,
wherein “X” represents any of the 20 natural amino
acids, preferably Cysteine, Alanine or Serine.

In another embodiment, the SARS-CoV2 fragment fusion product expressed in the lumen or in the membrane of the OMVs is the fusion of Fragments 6, 5 and 4, consisting of the sequence SEQ ID NO:9 (Fragment 9) or a derivative thereof having at least 75%, preferably at least 85% sequence identity to SEQ ID NO:9:

(Fragment 9, SEQ ID NO: 9)
LLNKHIDAYKTFPGSDIPIGAGIXASYQTQTNSPRRARSVASQSIIAYGS
DISTEIYQAGSTPXNGVEGFNXYFPLQSYGFQPINGVGYQPYR,
wherein “X” represents any of the 20 natural amino
acids, preferably Cysteine, Alanine or Serine.

In another embodiment, the SARS-CoV2 fragment fusion product expressed in the lumen or in the membrane of the OMVs is the fusion of Fragments 6, 5 and 2, and consists of the sequence SEQ ID NO:10 (Fragment 10) or a derivative thereof having at least 75%, preferably at least 85% sequence identity to SEQ ID NO:10:

(Fragment 10, SEQ ID NO: 10)
LLNKHIDAYKTFPGSDIPIGAGIXASYQTQTNSPRRARSVASQSIIAYGS
SNNLDSKVGGNYNYLYRLFRKSNLKPFERDISTEIYQAGSTPXNGVEGFN
XYFPLQSYGFQPTNGVGYQPYR,
wherein “X” represents any of the 20 natural amino
acids, preferably Cysteine, Alanine or Serine.

In another embodiment, the SARS-CoV2 fragment fusion product expressed in the lumen or in the membrane of the OMVs is the fusion of Fragments 5, 6 and 4, and consists of the sequence SEQ ID NO:11 (Fragment 11) or a derivative thereof having at least 75%, preferably at least 85% sequence identity to SEQ ID NO:11:

(Fragment 11, SEQ ID NO: 11)
DIPIGAGIXASYQTQTNSPRRARSVASQSIIAYGSLLNKHIDAYKTFPGS
DISTEIYQAGSTPXNGVEGFNXYFPLQSYGFQPTNGVGYQPYR,
wherein “X” represents any of the 20 natural amino
acids, preferably Cysteine, Alanine or Serine.

In another embodiment, the SARS-CoV2 fragment fusion product expressed in the lumen or in the membrane of the OMVs is the fusion of Fragments 5, 6 and 2, and consists of the sequence SEQ ID NO:12 (Fragment 12) or a derivative thereof having at least 75%, preferably at least 85% sequence identity to SEQ ID NO:12:

(Fragment 12, SEQ ID NO: 12)
DIPIGAGIXASYQTQTNSPRRARSVASQSIIAYGSLLNKHIDAYKTFPGS
SNNLDSKVGGNYNYLYRLFRKSNLKPFERDISTEIYQAGSTPXNGVEGFN
XYFPLQSYGFQPTNGVGYQPYR,
wherein “X” represents any of the 20 natural amino
acids, preferably Cysteine, Alanine or Serine.

In another embodiment, the SARS-CoV2 protein fragment expressed in the lumen or in the membrane of the OMVs is the SARS-CoV2 Spike polypeptide consisting of the sequence SEQ ID NO:13 (Fragment 13) or a derivative thereof having at least 75%, preferably at least 85% sequence identity to SEQ ID NO:13:

(Fragment 13, SEQ ID NO: 13)
VFVSNGTHWFVTQRNFY.

Fragments from 1 to 13 can be expressed as single copies or multiple copies in the lumen or in the membrane of OMVs by fusing them to appropriate leader sequences which promote the translocation of the polypeptides into the periplasmic space or their insertion into the outer membrane as lipidated polypeptides. Typical leader sequences are the OmpA leader sequence and the Lpp leader sequences but any other periplasmic and lipoprotein leader sequence can be conveniently used.

In a preferred embodiment of the invention, fragments 1 to 13 are expressed in the OMV membrane as fusions to the C-terminus of the Staphylococcus aureus lipoprotein FhuD2.

In a further aspect, the invention relates to a recombinant expression vector comprising an expression cassette containing a polynucleotide encoding a SARS-CoV2 protein, fragment thereof or multiple fragment fusion product as herein disclosed, operably linked to regulatory elements for controlling protein expression in a prokaryotic cell. Preferably the expression vector is a plasmid which include appropriate sequences for an effective transcription and translation of the cloned gene/gene fusion. Typically, pET21b-derived plasmids are used as the expression vector. However, any other plasmid backbone suitable for bacterial gene expression known in the art can be used. Suitable plasmids include pGEX, pUC19, pALTR, pET, pQE, pLEX, pHAT or any other plasmid vector that is capable of replication in Gram-negative bacteria.

The OMVs can be produced from bacterial cultures by biomass treatment with mild detergents, such as deoxycolate. Alternatively, if hypervesiculating strains are used, OMVs can be directly collected from the culture supernatant without detergent treatment.

The bacterial vesicles can conveniently be separated from whole bacterial culture by filtration e.g. through a 0.22 μm filter. Bacterial filtrates may be clarified by centrifugation, for example high speed centrifugation (e.g. 200,000×g for about 2 hours). Another useful process for OMV preparation is described in WO2005/004908 and involves ultrafiltration on crude OMVs, instead of high speed centrifugation. The process may involve a step of ultracentrifugation after the ultrafiltration takes place. A simple process for purifying bacterial vesicles comprises: (i) a first filtration step in which the vesicles are separated from the bacteria based on their different sizes, and (ii) tangential flow filtration using membranes that retain vesicles, thus allowing their concentration.

In a further embodiment, the invention provides an immunogenic composition comprising a bacterial outer membrane vesicle as herein disclosed, together with pharmaceutical acceptable vehicles and excipients.

The composition of the invention is in a suitable administration form and it is preferably in the form of a vaccine. Vaccines according to the invention may either be prophylactic or therapeutic. Pharmaceutical compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system. The amount of OMVs in the compositions of the invention may generally be between 10 and 500 μg, preferably between 25 and 200 μg, and more preferably about 50 idg or about 100 μg.

Compositions of the invention may be prepared in various liquid forms. For example, the compositions may be prepared as injectables, either as solutions or suspensions. The composition may be prepared for pulmonary administration e.g. by an inhaler, using a fine spray. The composition may be prepared for nasal, aural or ocular administration e.g. as spray or drops, and intranasal vesicle vaccines, as known in the art. Other suitable administration routes include intramuscular, oral, parenteral, transmucosal, or intradermal administration. Injection may be via a needle (e.g. a hypodermic needle), but needle-free injection may alternatively be used.

The OMVs and the immunogenic compositions according to the invention are conveniently used for the prophylaxis or therapy of SARS-CoV2 infection by stimulating an immune response against portions/domains of SARS-CoV2 proteins.

### DESCRIPTION OF THE FIGURES

FIG. 1

Cloning strategy used to fuse SARS-CoV2 Fragment 4 epitope to the C-terminus of FhuD2—The DNA sequence coding for one copy of SARS-CoV2 Fragment 4 was PCR amplified from pUC-SARS Cov2 RBD plasmid. The pET-FhuD2 vector was linearized by PCR amplification using the divergent primers nohis flag F/FhuD2-V-R. Finally, the PCR products were mixed together and used to transform HK-100 competent cells, obtaining plasmids pET-FhuD2-Fragment 4.

FIG. 2

Representation of pET-FhuD2-Fragment 4 plasmid—The DNA sequence refers to the 3′ end of the gene fusion encoding one copy of SARS-Cov2 Fragment 4

FIG. 3

Cloning strategy used to fuse SARS-CoV2 Fragment 2 epitope to the C-terminus of FhuD2—The DNA sequence coding for one copy of SARS-CoV2 Fragment 2 were PCR amplified from pUC-SARS Cov2 RBD plasmid. In particular two domains were separately amplified by PCR and combined together to generate SARS-CoV2 Fragment 2. The pET-FhuD2 vector was linearized by PCR amplification using the divergent primers nohis flag F/FhuD2-V-R. Finally, the PCR products were mixed together and used to transform HK-100 competent cells, obtaining plasmids pET-FhuD2-Fragment 4.

FIG. 4

Representation of pET-FhuD2-Fragment 2 plasmid—The DNA sequence refers to the 3′ end of the gene fusion encoding one copy of SARS-Cov2 Fragment 2

FIG. 5

Cloning strategy used to fuse two copies of SARS-CoV2 Fragment 5 epitope to the C-terminus of FhuD2—The DNA sequence coding two copies of SARS-CoV2 Fragment 5 separated by a dipeptide Gly-Ser were assembled by PCR oligo annealing. The pET-FhuD2 vector was linearized by PCR amplification using the divergent primers nohis flag F/FhuD2-V-R. Finally, the PCR products were mixed together and used to transform HK-100 competent cells, obtaining plasmids pET-FhuD2-Fragment 5.

FIG. 6

Representation of pET-FhuD2-Fragment 5 plasmid—The DNA sequence refers to the 3′ end of the gene fusion encoding one copy of SARS-Cov2 Fragment 5.

FIG. 7

SDS-PAGE of OMVs from BL21(DE3)/A60 strains expressing different SARS-CoV2-derived fragments fused to FhuD2 protein—OMVs were purified from BL21(DE3)/460 recombinant strains, each expressing one specific FhuD2 fusion (FhuD2-Fragment 4, FhuD2-Fragment 2, FhuD2-Fragment 5). Total OMV proteins were separated by SDS-PAGE and stained with Coomassie brilliant blue. Asterisks highlight the bands corresponding to recombinant antigens.

FIG. 8

Antibody titers in mice immunized with OMVs from recombinant strains expressing FhuD2-Fragment 4 and FhuD2-Fragment 2—A) Schematic representation of immunization schedule in CD1 mice. B) ELISA plates were coated with recombinant RBD purified from E. coli, recombinant RBD purified from eukaryotic cells, full length Spike proteins purified from eukaryotic cells and the titers of antibodies recognizing coated proteins were analyzed in sera from mice immunized with “Empty” OMVs (control), FhuD2-fragment 2-OMVs, and FhuD2-Fragment 4-OMVs.

FIG. 9

Neutralization of SARS-CoV-2 pseudotypes infectivity by sera from mice immunized with engineered OMVs—HIV-1 vectors pseudotyped with the SARS-CoV-2 spike glycoprotein were mixed with increasing concentration of sera derived from mice vaccinated with the indicated spike fragments or from control mice, and used to infect reporter cells expressing the ACE2 receptor. Residual infection was calculated considering the amount of fluorescent infected cells in the no-serum sample as 100%. Shown are mean values and standard variations (n=3).

### DETAILED DESCRIPTION OF THE INVENTION

### Engineering of OMVs with Portions/Domains of SARS-CoV2 Proteins (Fragments 2, 4, and 5)

### Construction of FhuD2-Fragment 4 Fusion

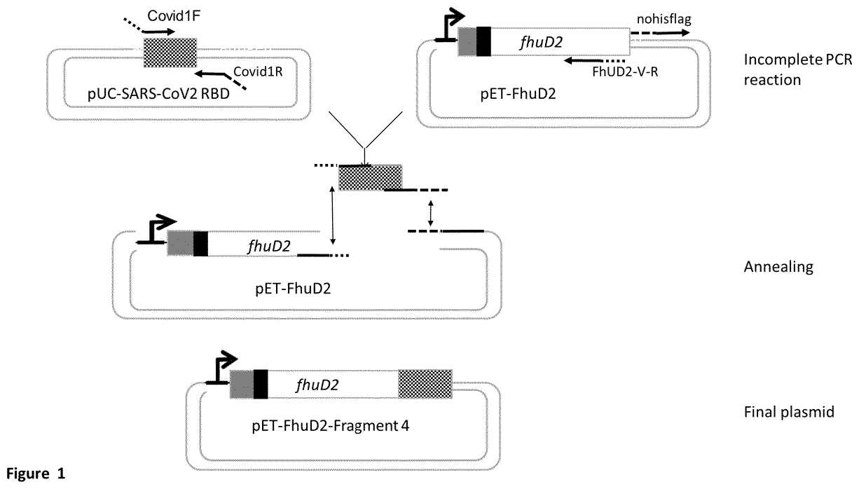

To engineer OMVs with the portion of the SARS-CoV2 Spike protein named “Fragment 4”, the strategy depicted in FIG. 1 was used. First of all, the DNA fragment coding the entire RBD domain was chemically synthesized (SEQ ID NOs:31 and 32) and inserted in pUC plasmid, thus creating plasmid pUC-SARS-Cov2 (geneArt). Subsequently,

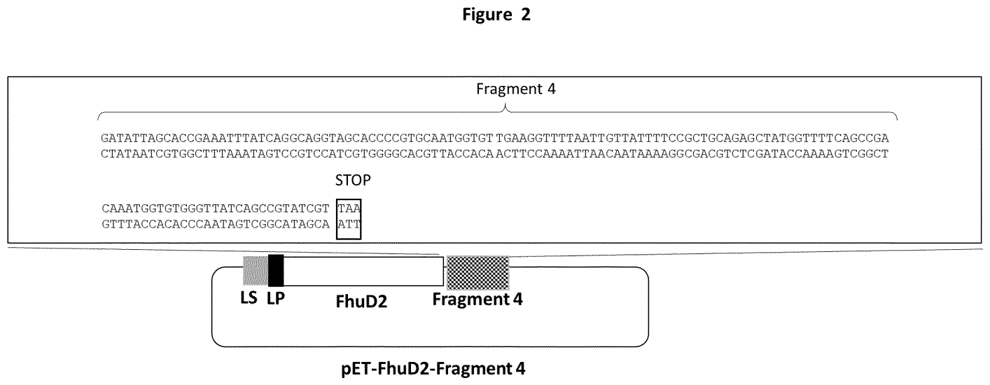

SARS-Cov2-Fragment 4 was amplified by PCR from the pUC-SARS-Cov2 with Covid1F/Covid1R primers. These primers were designed to generate extremities complementary to 3′ end of the FhuD2 gene present in pET-FhuD2 plasmid (Irene et al. (2019) PNAS, 116, 21780-21788). In parallel, pET-FhuD2 plasmid was linearized by PCR amplification using the divergent primers nohis flag F/FhuD2-V-R. Finally, the PCR products were mixed together and used to transform HK-100 competent cells, obtaining pET-FhuD2-Fragment 4 (FIG. 2). The accuracy of the final plasmid was verified by sequence analysis (SEQ ID NOs:33 and 34)

### Construction of FhuD2-Fragment 2 Fusion

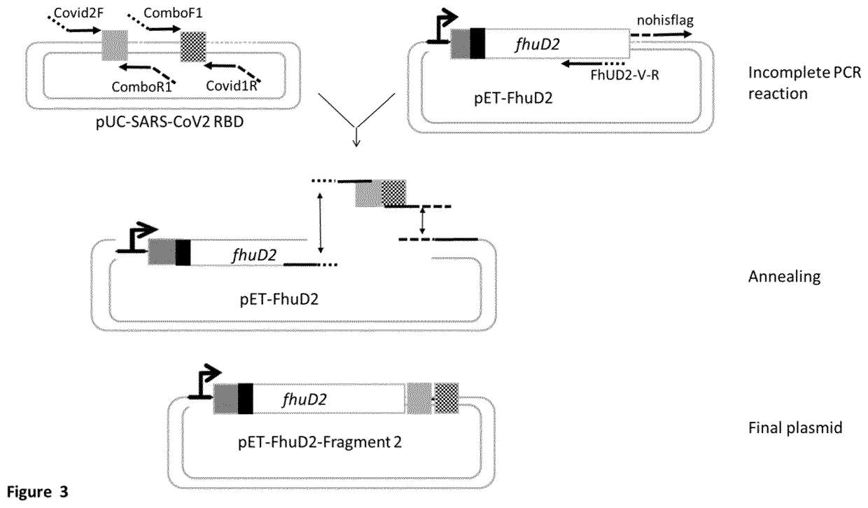

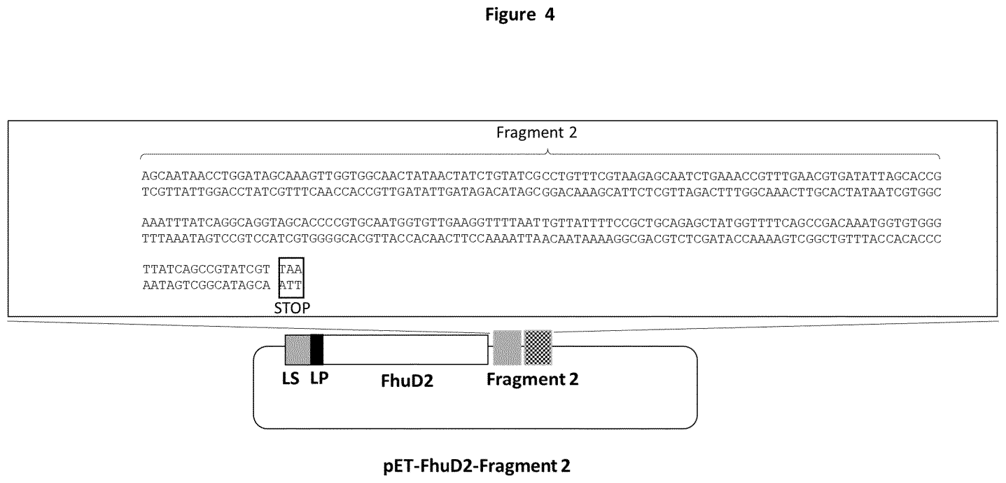

To engineer OMVs with the portion of the SARS-CoV2 Spike protein named “Fragment 2”, the strategy depicted in FIG. 3 was used. First of all, the SARS-Cov2 RBD “chain 1” and the SARS-Cov2 RBD “chain 2” were amplified by PCR from pUC-SARS-Cov2 plasmid with ComboF1/Covid1R and Covid2F/ComboR1 primers, respectively. These primers were designed to generate extremities complementary between the two chains. Then using 1 ng of PCR products of the two reactions as template, the SARS-Cov2-Fragment 2 was amplified using Covid2F/CovidlR primers. These primers were designed to generate extremities complementary to 3′ end of the FhuD2 gene present pET-FhuD2 plasmid. In parallel, pET-FhuD2 plasmid was linearized by PCR amplification using the divergent primers nohis flag F/FhuD2-V-R. Finally, the PCR products were mixed together and used to transform HK-100 competent cells, obtaining pET-FhuD2-Fragment 2 (FIG. 4). The accuracy of the final plasmid was verified by sequence analysis (SEQ ID NOs:35 and 36).

### Construction of FhuD2-Fragment 5 Fusion

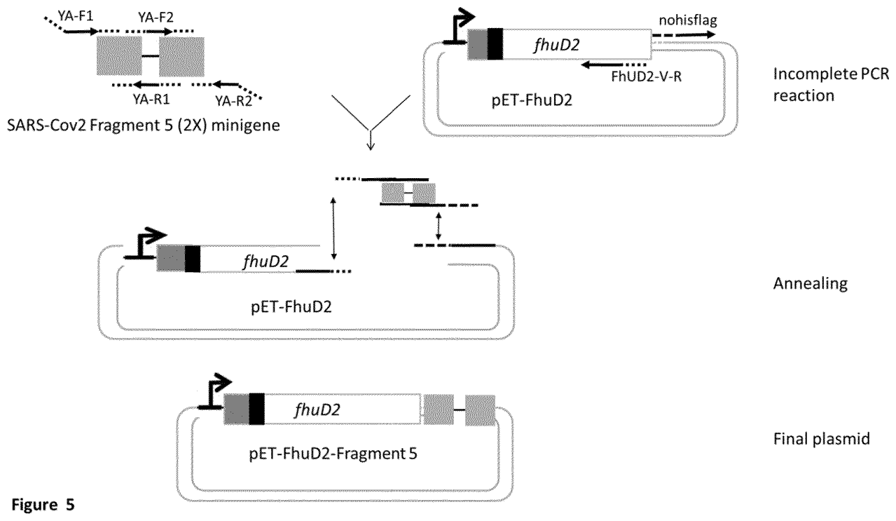

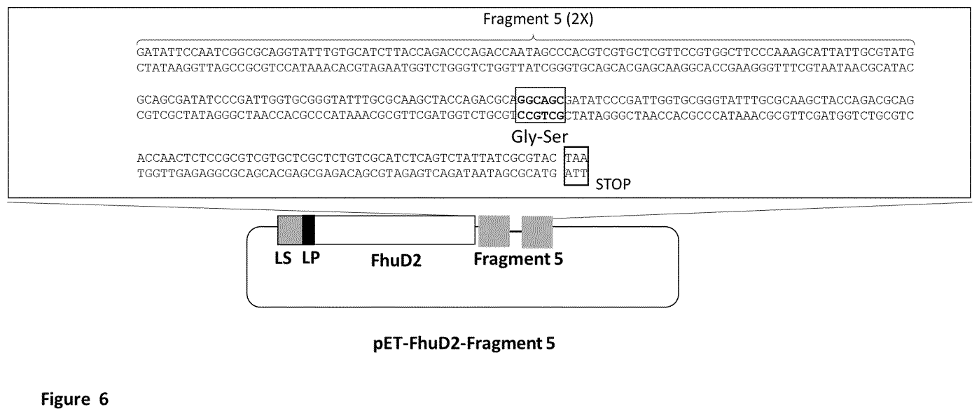

To engineer OMVs with the portion of the SARS-CoV2 Spike protein named “Fragment 5”, the strategy depicted in FIG. 5 was used. Two copies of SARS-Cov2 Fragment 5 domain were fused to the C-terminus of the FhuD2 lipoprotein. First of all, the minigene carrying two copies of SARS-Cov2 Fragment 5 separated by the Gly-Ser dipeptide were annealed by PCR with the oligo Ya-F1, Ya-R1, Ya-F2 and Ya-R2 (see table). Then the minigene was amplified by PCR with Ya-StaF/Yax2R primers. These primers were designed to generate extremities complementary to the pET-FhuD2 plasmid. This vector was linearized by PCR amplification using the divergent primers nohis flag F/FhuD2-V-R. Finally, the PCR products were mixed together and used to transform HK-100 competent cells, obtaining pET-FhuD2-Fragment 5 plasmid (FIG. 6). The accuracy of the final plasmid was verified by sequence analysis (SEQ ID NOs:37 and 38).

### Construction of FhuD2-Fragment 5 Fusion

TABLE 1
Oligonucleotide primers used for plasmids and genes preparation
pET-FhuD2, pET-15b-His tag-TEV

| NAME | SEQUENCE |
|---|---|
| nohis flag | CATCACCATCACCATCACGATTACA (SEQ ID NO: 14) |
| Lpp-R-plasmid | GCTGGAGCAACCTGCCAGCAGAG (SEQ ID NO: 15) |
| EK1084 | GCCCTGGAAGTACAGGTTTTC (SEQ ID NO: 16) |
| EK1085 | CGCGACTTAATTCTAGCATAAC (SEQ ID NO: 17) |

Oligo for Fragment 5 minigene

| | |
|---|---|
| Ya-F1 | GATATTCCAATCGGCGCAGGTATTTGTGCATCTTACCAGACCCAGACCAATAGCCCA (SEQ ID NO: 18) |
| Ya-R1 | ATACGCAATAATGCTTTGGGAAGCCACGGAACGAGCACGACGTGGGCTATTGGTCTG (SEQ ID NO: 19) |
| Ya-F2 | AGCATTATTGCGTATGGCAGCGATATCCCGATTGGTGCGGGTATTTGCGCAAGCTACCAGACGCA (SEQ ID |
| | NO: 20) |
| Ya-R2 | GTACGCGATAATAGACTGAGATGCGACAGAGCGAGCACGACGCGGAGAGTTGGTCTGCGTCTGGTAGCTT |
| | (SEQ ID NO: 21) |

pET-FhuD2-Fragment 4, pET-FhuD2- Fragment 2, pET-15-RBD plasmids, pET-FhuD2-Fragment 5

| NAME | SEQUENCE |
|---|---|
| Covid1F | TAATTAAAGCTGCAAAAGATATTAGCACCGAAATTTATCAGGC (SEQ ID NO: 22) |
| Covid1R | GATGGTGATGGTGATGTTAACGATACGGCTGATAACCCAC (SEQ ID NO: 23) |
| ComboF1 | CCGTTTGAACGTGATATTAGCACCGAAATTTATCAGG (SEQ ID NO: 24) |
| Covid2F | TAATTAAAGCTGCAAAAAGCAATAACCTGGATAGCAAAG (SEQ ID NO: 25) |
| ComboR1 | ACGTTCAAACGGTTTCAGATTGCTCTTACGAAAC (SEQ ID NO: 26) |
| Covidpet-F | CTGTACTTCCAGGGCCCGAATATCACCAATCTGTGTCC (SEQ ID NO: 27) |
| Covidpet-R | TAGAATTAAGTCGCGTTATTATTCCAGTGTCTGCGGATCAC (SEQ ID NO: 28) |
| Ya-StaF | TAATTAAAGCTGCAAAAGATATTCCAATCGGCGCAGGTA (SEQ ID NO: 29) |
| Ya-2xR | GATGGTGATGGTGATGTTAGTACGCGATAATAGACTGAGAT (SEQ ID NO: 30) |

### Expression of FhuD2 SARS-CoV2 Fusions in OMVs

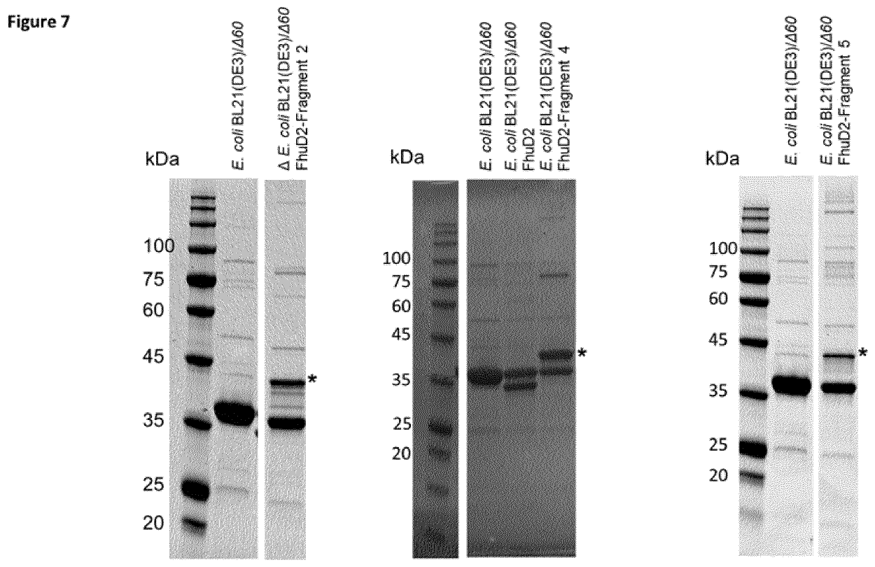

The recombinant plasmids, generated as previously described, encoding the selected SARS-CoV2 fragments fused to the FhuD2 lipoprotein were used to transform E. coli BL21(DE3)/460 strain. Bacteria were grown in LB medium and when the cultures reached an OD600 value of 0.5, IPTG was added at 0.1 mM final concentration. After two additional hours of growth at 37° C., vesicles were purified from culture supernatants by using ultrafiltration coupled to ultracentrifugation. More specifically, OMVs were collected from culture supernatants by filtration through a 0.22 μm pore size filter (Millipore) and by high-speed centrifugation (200,000×g for 2 hours). Pellets containing OMVs were finally suspended in PBS. The presence of FhuD2 fusions was analyzed by SDS-PAGE. As shown in FIG. 7, the three SARS-CoV2 FhuD2 fusion proteins could be visualized by Coomassie Blue staining and compartmentalized in OMVs.

### Engineered OMVs Carrying Recombinant FhuD2 Fusion Proteins Induce High Titers of SARS-CoV2-Specific Antibodies

To test whether OMVs purified from recombinant strains expressing FhuD2-Fragment 4 and FhuD2-Fragment 2 fusions were capable of inducing antibodies recognizing SARS-CoV2 S protein, groups of CD1 mice were i.p. immunized three times at two-week intervals with 10 μg of OMVs+Alum (2 mg/ml). Blood samples were collected seven days after the second dose (post2) or the third dose (post3) and the level of specific IgGs specific for the RBD domain and the full length S protein were detected by ELISA. In particular, ELISA plates were coated with (0.511 g/well) with either recombinant RBD domain expressed and purified from E. coli, or RBD domain and full length S protein purified from HEK293 cells. RBD domain (sequence 1) was inserted into pET-15-His-TEV plasmid and purified from E. coli BL21. Bacteria were grown in LB medium and when the cultures reached an OD600 value of 0.5, IPTG was added at 1 mM final concentration. After two additional hours of growth at 37° C. bacterial pellet was collect by centrifugation at 4000 g for 10 min at 4° C. Pellet was incubated in B-per buffer (Thermo Fhisher) for 30 min on ice and then centrifuged at 14000 rpm for 30 min at 4° C. The pellet was dissolved in 6M Guanidine loaded on a pre equilibrated IMAC Sepharose® 6 Fast Flow column. His-tagged RBD was eluted with a solution of 300 mM Imidazole and 6M Urea.

Coating of ELISA plates was carried out by incubating plates overnight incubation at 4° C. with 100 μl/well of proteins (5 μg/ml). Subsequently, wells were washed three times with PBST (0.05% Tween 20 in PBS, pH 7.4), incubated with 100 μl of 1% BSA in PBS for 1 h at room temperature and washed again three times with PB ST. Serial dilutions of serum samples from immunized mice (sera from mice immunized with E. coli BL21(DE3)/460-derived “empty” OMVs was used as negative control) in PBST containing 1% BSA were added to the plates, incubated 2 h at 37° C., and washed three times with PBST. Then 100 μl/well of 1:2.000 diluted, alkaline phosphatase-conjugated goat anti-mouse IgGs were added and left for 1 h at 37° C. After three PBST washes, bound alkaline phosphatase-conjugated antibodies were detected by adding 100 μl/well of 1 mg/ml para-nitrophenyl-phosphate disodium hexahydrate (Sigma—Aldrich) in 1M diethanolamine buffer (pH 9.8). After 30 minutes incubation at room temperature, the reaction was stopped with 100 μl 7% EDTA and substrate hydrolysis was analyzed at 405 nm in a microplate spectrophotometer.

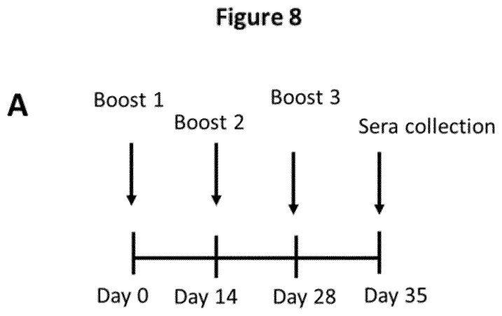

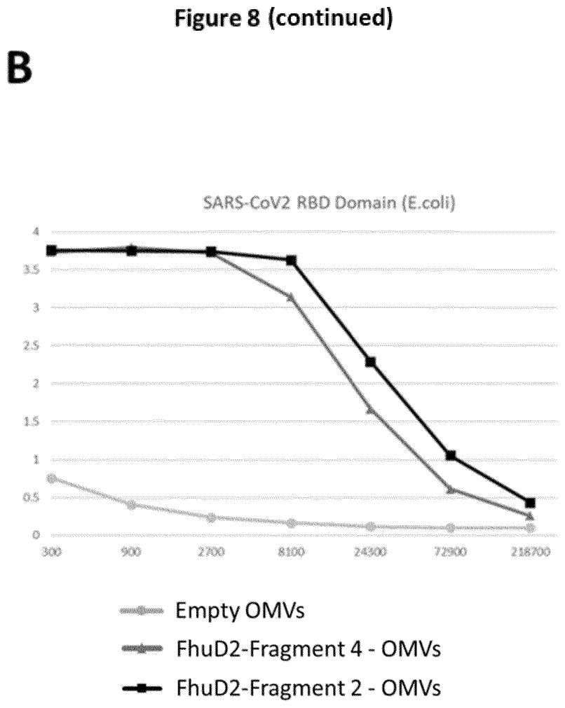

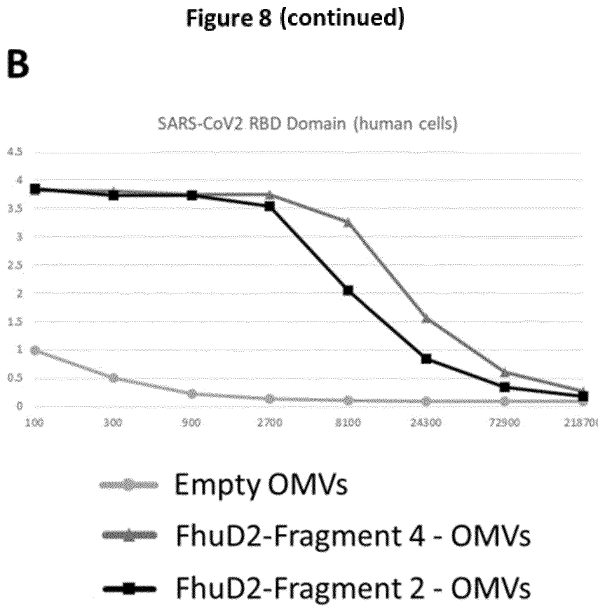

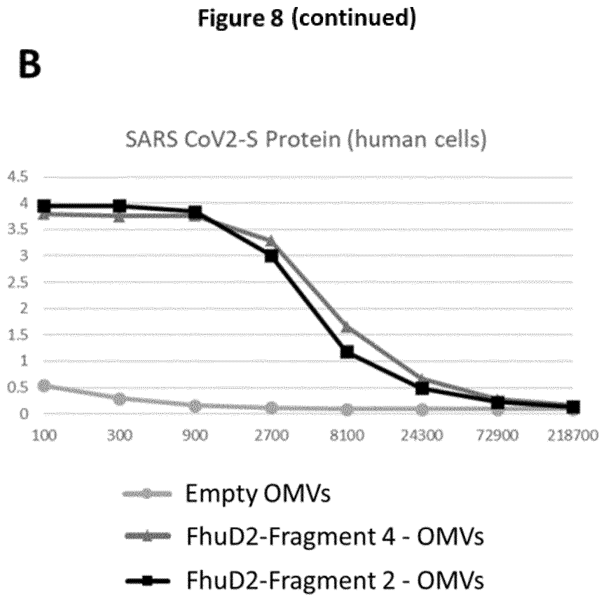

As shown in FIG. 8, OMVs carrying FhuD2-Fragment 2 and FhuD2-Fragment 4 fusion proteins were able to induce high specific IgG titers in immunized mice against SARS-CoV2 antigens. Importantly, both sera were able recognized with similar affinities denatured RBD purified from E. coli and soluble RBD and native full-length S protein purified from cell cultures.

### Engineered OMVs Carrying Recombinant FhuD2 Fusion Proteins Induce SARS-CoV2-Specific Antibodies Which Inhibit Viral Entry in Human Cells

The neutralization capacity of animal and human sera against SARS-CoV-2 is tested using a pseudotype assay, similar to those already in use for SARS-CoV 1 and other respiratory viruses2. Pseudotyping is a technique that allows the construction of biologically safe chimeric viruses (handled in BSL-2) to study and quantify entry of viruses that would otherwise require handling in highly restrictive containment (BSL-3 and BSL-4). Since coronaviruses such as SARS-COV-2 only require the spike glycoprotein (S) to attach the cognate receptor (ACE2) and to gain access to the target cells, it is sufficient to display the spike on recombinant retroviral vectors to obtain virus-like particles (VLPs) that enter the target cells with a mechanism indistinguishable to that used by bona-fide SARS-CoV-2 virions. This is an ideal system to assess the neutralization activity elicited in vaccinees and convalescent patients, being amenable to studies which require high throughput analysis, such as clinical and epidemiological investigations.

Recombinant HIV-1 vectors are pseudotyped by expressing, in vector-producing cells, the ORF which encodes the spike glycoprotein derived from the SARS-CoV-2 reference genome (accession n. NC_045512). Spike proteins derived from other isolates can be similarly used. To improve the efficiency of pseudotyping, a mutation was introduced in the cytoplasmic domain of the glycoprotein in order to insert a premature stop codon to prevent translation of the C-terminal 20 amino acids, which code for a reticulum retention signal that would otherwise interfere with the transport of the glycoprotein through the Golgi to the cell surface, where HIV-1 budding primarily occurs. Vectors will be used to transduce a reporter cell line which was constructed to favour robust detection with a multiplate fluorescence imager (Perkin Elmer Ensight). The reporter cell line is a derivatine of TZM-bl-ZsGreen-nls derived in our laboratories (Rosa, A. et al. (2015) Nature 526, 212-7), which was further engineered to ectopically express human ACE2 and the protease TMPRS S2 required for the processing of the spike glycoprotein in the endosomal compartment (Hoffmann, M et al. (2020) Cell (2020) doi: 10.1016/j.cell.2020. 02.052). The cell line contains an HIV-1 Tat-inducible nuclear-localized ZsGreen fluorescent proteins which generate a discrete nuclear fluorescence robustly quantifiable by the imager. A lentiviral genetic vector encoding Tat will be packaged in the pseudotyped VLPs to deliver the transactivator to target cells in the absence of any other HIV viral genes, making this a biologically safe system.

VLPs, at a concentration adjusted to produce a transduction not higher than 10% of the cells in the monolayer, are incubated with serially diluted sera for 30 minutes at room temperature. The complexes are added to reporter cells and incubated at 37° C. for a further 48 hours before quantification with cell imager. Neutralization is measured by calculating the residual infectivity of treated VLPs samples. Fitted sigmoidal curves and IC50 are obtained using Prism (Graphpad) with the least square variable slope method and using the dose-normalized response protocol.

Sera derived from mice immunized with OMVs expressing FhuD2-Fragment 2 and FhuD2-Fragment 4 were serially diluted as indicated and combined with pseudotyped vectors. After a 30 minutes incubation at room temperature, the vector-sera complexes were added to target cell monolayers in 96-well plates in triplicate. GFP transduction was scored using Perkin Elmer Ensight plate reader/imager by enumerating the fluorescent cells per each well. The graph shows the residual transduction per each serum dilution considering the amount of fluorescent cells in the no-serum sample as 100%. Shown are mean values and standard variations (n=3).

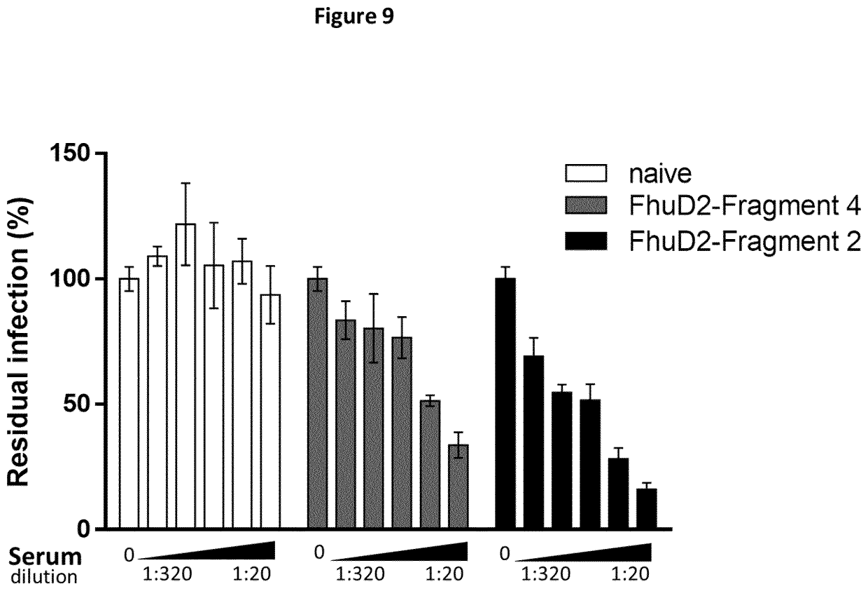

The data shown in FIG. 9 demonstrate that sera from immunized mice efficiently neutralize the SARS-CoV-2 spike glycoprotein with an IC50, calculated from fitted sigmoidal curves with the least square variable slope method, of 1:36 and 1:110 for #1 and #2 respectively.

SEQUENCES
SARS-Cov2 RBD
DNA sequence
CCGAATATCACCAATCTGTGTCCGTTTGGTGAAGTTTTTAATGCAACCCGTTTTGCAAGC
GTTTATGCCTGGAATCGTAAACGTATTAGCAATTGCGTTGCCGATTATAGCGTTCTGTAT
AATAGCGCAAGCTTCAGCACCTTTAAATGCTATGGTGTTAGCCCGACCAAACTGAATGAT
CTGTGTTTTACCAATGTGTATGCCGATAGCTTTGTGATTCGTGGTGATGAAGTTCGTCAG
ATTGCACCGGGTCAGACCGGTAAAATTGCAGATTATAACTATAAACTGCCGGATGATTTT
ACGGGTTGTGTTATTGCATGGAATAGCAATAACCTGGATAGCAAAGTTGGTGGCAACTAT
AACTATCTGTATCGCCTGTTTCGTAAGAGCAATCTGAAACCGTTTGAACGTGATATTAGC
ACCGAAATTTATCAGGCAGGTAGCACCCCGTGCAATGGTGTTGAAGGTTTTAATTGTTAT
TTTCCGCTGCAGAGCTATGGTTTTCAGCCGACAAATGGTGTGGGTTATCAGCCGTATCGT
GTTGTTGTTCTGTCATTTGAACTGCTGCATGCACCGGCAACCGTTTGTGGTCCGAAAAAA
AGTACCAATCTGGTGAAAAACAAGTGCGTGAACTTTAACTTTAATGGTCTGACCGGCACC
GGTGTTCTGACCGAAAGCAATAAAAAGTTTCTGCCGTTTCAGCAGTTTGGCCGTGATATT
GCAGATACCACCGATGCAGTTCGTGATCCGCAGACACTGGAATAA (SEQ ID NO: 31)
Amino acid sequence
PNITNLCPFGEVFNATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGNY
NYLYRLERKSNLKPFERDISTEIYQAGSTPCNGVEGENCYFPLQSYGFQPTNGVGYQPYR
VVVLSFELLHAPATVCGPKKSTNLVKNKCVNFNFNGLTGTGVLTESNKKFLPFQQFGRDI
ADTTDAVRDPQTLE (SEQ ID NO: 32)
pET-FhuD2-Fragment 4
DNA sequence
ATGATGAAAGCTACTAAACTGGTACTGGGCGCGGTAATCCTGGGTTCTACTCTGCTGGCA
GGTTGCGGGAACCAAGGTGAAAAAAATAACAAAGCTGAAACTAAATCTTATAAAATGGAC
GATGGCAAAACGGTAGATATTCCGAAAGACCCTAAACGCATTGCAGTAGTTGCGCCAACA
TATGCTGGTGGACTTAAAAAATTAGGTGCAAACATTGTAGCTGTAAATCAACAAGTCGAT
CAAAGCAAAGTATTAAAAGATAAATTTAAAGGTGTTACAAAAATTGGTGATGGCGATGTA
GAAAAAGTTGCTAAAGAAAAGCCAGATTTAATTATTGTATACTCTACTGACAAAGATATT
AAAAAATATCAAAAAGTAGCACCAACAGTAGTTGTTGACTATAATAAGCATAAATATTTA
GAACAACAAGAAATGTTAGGGAAAATTGTTGGTAAAGAAGATAAAGTAAAAGCTTGGAAG
AAAGATTGGGAAGAAACAACTGCTAAAGACGGTAAAGAAATTAAAAAAGCAATTGGACAA
GATGCAACAGTGTCATTGTTTGATGAATTTGATAAAAAATTATACACTTACGGCGATAAC
TGGGGTCGTGGTGGAGAAGTATTATATCAAGCATTTGGTTTGAAAATGCAACCAGAACAA
CAAAAGTTAACTGCAAAAGCAGGTTGGGCTGAAGTGAAACAAGAAGAAATTGAAAAATAT
GCTGGTGATTACATTGTGAGTACAAGTGAAGGTAAACCTACACCAGGATACGAATCAACA
AACATGTGGAAGAATTTGAAAGCTACTAAAGAAGGACATATTGTTAAAGTTGATGCTGGT
ACATACTGGTACAACGATCCTTATACATTAGATTTCATGCGTAAAGATTTAAAAGAAAAA
TTAATTAAAGCTGCAAAAGATATTAGCACCGAAATTTATCAGGCAGGTAGCACCCCGTGC
AATGGTGTTGAAGGTTTTAATTGTTATTTTCCGCTGCAGAGCTATGGTTTTCAGCCGACA
AATGGTGTGGGTTATCAGCCGTATCGT (SEQ ID NO: 33)
Amino acid sequence
MMKATKLVLGAVILGSTLLAGCGNQGEKNNKAETKSYKMDDGKTVDIPKDPKRIAVVAPT
YAGGLKKLGANIVAVNQQVDQSKVLKDKFKGVTKIGDGDVEKVAKEKPDLIIVYSTDKDI
KKYQKVAPTVVVDYNKHKYLEQQEMLGKIVGKEDKVKAWKKDWEETTAKDGKEIKKAIGQ
DATVSLFDEFDKKLYTYGDNWGRGGEVLYQAFGLKMQPEQQKLTAKAGWAEVKQEEIEKY
AGDYIVSTSEGKPTPGYESTNMWKNLKATKEGHIVKVDAGTYWYNDPYTLDFMRKDLKEK
LIKAAKDISTEIYQAGSTPCNGVEGFNCYFPLQSYGFQPTNGVGYQPYR
(SEQ ID NO: 34)
pET-FhuD2-Fragment 2
DNA sequence
ATGATGAAAGCTACTAAACTGGTACTGGGCGCGGTAATCCTGGGTTCTACTCTGCTGGCA
GGTtgcGGGAACCAAGGTGAAAAAAATAACAAAGCTGAAACTAAATCTTATAAAATGGAC
GATGGCAAAACGGTAGATATTCCGAAAGACCCTAAACGCATTGCAGTAGTTGCGCCAACA
TATGCTGGTGGACTTAAAAAATTAGGTGCAAACATTGTAGCTGTAAATCAACAAGTCGAT
CAAAGCAAAGTATTAAAAGATAAATTTAAAGGTGTTACAAAAATTGGTGATGGCGATGTA
GAAAAAGTTGCTAAAGAAAAGCCAGATTTAATTATTGTATACTCTACTGACAAAGATATT
AAAAAATATCAAAAAGTAGCACCAACAGTAGTTGTTGACTATAATAAGCATAAATATTTA
GAACAACAAGAAATGTTAGGGAAAATTGTTGGTAAAGAAGATAAAGTAAAAGCTTGGAAG
AAAGATTGGGAAGAAACAACTGCTAAAGACGGTAAAGAAATTAAAAAAGCAATTGGACAA
GATGCAACAGTGTCATTGTTTGATGAATTTGATAAAAAATTATACACTTACGGCGATAAC
TGGGGTCGTGGTGGAGAAGTATTATATCAAGCATTTGGTTTGAAAATGCAACCAGAACAA
CAAAAGTTAACTGCAAAAGCAGGTTGGGCTGAAGTGAAACAAGAAGAAATTGAAAAATAT
GCTGGTGATTACATTGTGAGTACAAGTGAAGGTAAACCTACACCAGGATACGAATCAACA
AACATGTGGAAGAATTTGAAAGCTACTAAAGAAGGACATATTGTTAAAGTTGATGCTGGT
ACATACTGGTACAACGATCCTTATACATTAGATTTCATGCGTAAAGATTTAAAAGAAAAA
TTAATTAAAGCTGCAAAAAGCAATAACCTGGATAGCAAAGTTGGTGGCAACTATAACTAT
CTGTATCGCCTGTTTCGTAAGAGCAATCTGAAACCGTTTGAACGTGATATTAGCACCGAA
ATTTATCAGGCAGGTAGCACCCCGTGCAATGGTGTTGAAGGTTTTAATTGTTATTTTCCG
CTGCAGAGCTATGGTTTTCAGCCGACAAATGGTGTGGGTTATCAGCCGTATCGT
(SEQ ID NO: 35)
Amino acid sequence
MMKATKLVLGAVILGSTLLAGCGNQGEKNNKAETKSYKMDDGKTVDIPKDPKRIAVVAPT
YAGGLKKLGANIVAVNQQVDQSKVLKDKFKGVTKIGDGDVEKVAKEKPDLIIVYSTDKDI
KKYQKVAPTVVVDYNKHKYLEQQEMLGKIVGKEDKVKAWKKDWEETTAKDGKEIKKAIGQ
DATVSLFDEFDKKLYTYGDNWGRGGEVLYQAFGLKMQPEQQKLTAKAGWAEVKQEEIEKY
AGDYIVSTSEGKPTPGYESTNMWKNLKATKEGHIVKVDAGTYWYNDPYTLDFMRKDLKEK
LIKAAKSNNLDSKVGGNYNYLYRLERKSNLKPFERDISTEIYQAGSTPCNGVEGFNCYFP
LQSYGFQPTNGVGYQPYR (SEQ ID NO: 36)
pET-FhuD2-Fragment 5
DNA sequence
ATGATGAAAGCTACTAAACTGGTACTGGGCGCGGTAATCCTGGGTTCTACTCTGCTGGCA
GGTtgcGGGAACCAAGGTGAAAAAAATAACAAAGCTGAAACTAAATCTTATAAAATGGAC
GATGGCAAAACGGTAGATATTCCGAAAGACCCTAAACGCATTGCAGTAGTTGCGCCAACA
TATGCTGGTGGACTTAAAAAATTAGGTGCAAACATTGTAGCTGTAAATCAACAAGTCGAT
CAAAGCAAAGTATTAAAAGATAAATTTAAAGGTGTTACAAAAATTGGTGATGGCGATGTA
GAAAAAGTTGCTAAAGAAAAGCCAGATTTAATTATTGTATACTCTACTGACAAAGATATT
AAAAAATATCAAAAAGTAGCACCAACAGTAGTTGTTGACTATAATAAGCATAAATATTTA
GAACAACAAGAAATGTTAGGGAAAATTGTTGGTAAAGAAGATAAAGTAAAAGCTTGGAAG
AAAGATTGGGAAGAAACAACTGCTAAAGACGGTAAAGAAATTAAAAAAGCAATTGGACAA
GATGCAACAGTGTCATTGTTTGATGAATTTGATAAAAAATTATACACTTACGGCGATAAC
TGGGGTCGTGGTGGAGAAGTATTATATCAAGCATTTGGTTTGAAAATGCAACCAGAACAA
CAAAAGTTAACTGCAAAAGCAGGTTGGGCTGAAGTGAAACAAGAAGAAATTGAAAAATAT
GCTGGTGATTACATTGTGAGTACAAGTGAAGGTAAACCTACACCAGGATACGAATCAACA
AACATGTGGAAGAATTTGAAAGCTACTAAAGAAGGACATATTGTTAAAGTTGATGCTGGT
ACATACTGGTACAACGATCCTTATACATTAGATTTCATGCGTAAAGATTTAAAAGAAAAA
TTAATTAAAGCTGCAAAAGATATTCCAATCGGCGCAGGTATTTGTGCATCTTACCAGACC
CAGACCAATAGCCCACGTCGTGCTCGTTCCGTGGCTTCCCAAAGCATTATTGCGTATGGC
AGCGATATCCCGATTGGTGCGGGTATTTGCGCAAGCTACCAGACGCAGACCAACTCTCCG
CGTCGTGCTCGCTCTGTCGCATCTCAGTCTATTATCGCGTAC (SEQ ID NO: 37)
Amino acid sequence
MMKATKLVLGAVILGSTLLAGCGNQGEKNNKAETKSYKMDDGKTVDIPKDPKRIAVVAPT
YAGGLKKLGANIVAVNQQVDQSKVLKDKFKGVTKIGDGDVEKVAKEKPDLIIVYSTDKDI
KKYQKVAPTVVVDYNKHKYLEQQEMLGKIVGKEDKVKAWKKDWEETTAKDGKEIKKAIGQ
DATVSLFDEFDKKLYTYGDNWGRGGEVLYQAFGLKMQPEQQKLTAKAGWAEVKQEEIEKY
AGDYIVSTSEGKPTPGYESTNMWKNLKATKEGHIVKVDAGTYWYNDPYTLDFMRKDLKEK
LIKAAKDIPIGAGICASYQTQTNSPRRARSVASQSIIAYGSDIPIGAGICASYQTQTNSP
RRARSVASQSIIAY (SEQ ID NO: 38)

1. A method of producing bacterial outer membrane vesicles (OMVs) carrying, in their lumen or membrane, a SARS-CoV2 protein, a SARS-CoV2 protein fragment, or a fusion product of (a) multiple copies of a SARS-CoV2 protein fragment or (b) multiple fragments from the same or different SARS-CoV2 protein(s), said method comprising:
(i) culturing a Gram-negative bacterium expressing a SARS-CoV2 protein or fragment or fusion product as above defined in the OMVs, in conditions suitable for vesiculation;
(ii) separating the OMVs from the bacterial culture,
and optionally,
(iii) purifying the OMVs.

2. The method of claim 1, wherein the SARS-CoV2 protein is the Spike protein or the Nucleocapsid (N) protein.

3. The method of claim 1, wherein the SARS-CoV2 protein fragments in the fusion product are separated by a spacer sequence of 2 to 10; 2 to 6; or 2 to 4 amino acids, which are preferably selected from Gly and Ser.

4. The method of claim 1, wherein the SARS-CoV2 protein fragment or fusion product is selected from the group consisting of:
(i) PNITNLXPFGEVFNATRFASVYAWNRKRISNXVADYSVLYNSASFSTFKX YGVSPTKLNDLXFTNVYADSFVIRGDEVRQIAPGQTGKIADYNYKLPDDFTGXVI AWNSNNLDS KVGGNYNYLYRLFRKSNLKPFERDISTEIYQAGSTPCNGVEGFNC YFPLQSYGFQPTNGVGYQPYRVVVLSFELLHAPATVXGPKKSTNLVKNKXVNFN FNGLTGTGVLTESNKKFLPFQQFGRDIADTTDAVRDPQTLE (Fragment 1, SEQ ID NO:1), or an immunogenic (poly)peptide having at least 75% sequence identity to SEQ ID NO:1;
(ii) SNNLDSKVGGNYNYLYRLFRKSNLKPFERDISTEIYQAGSTPCNGVEGFN CYFPLQSYGFQPTNGVGYQPYR (Fragment 2, SEQ ID NO:2), or an immunogenic (poly)peptide having at least 75% sequence identity to SEQ ID NO:2;
(iii) SNNLDSKVGGNYNYLYRLFRKSNLK (Fragment 3, SEQ ID NO:3), or an immunogenic (poly)peptide having at least 75% sequence identity to SEQ ID NO:3;
(iv) DISTEIYQAGSTPCNGVEGFNCYFPLQSYGFQPTNGVGYQPYR (Fragment 4, SEQ ID NO:4), or an immunogenic (poly)peptide having at least 75% sequence identity to SEQ ID NO:4;
(v) DIPIGAGIXASYQTQTNSPRRARSVASQSIIAY, (Fragment 5, SEQ ID NO:5), or an immunogenic (poly)peptide having at least 75% sequence identity to SEQ ID NO:5;
(vi) LLNKHIDAYKTFP (Fragment 6, SEQ ID NO:6), or an immunogenic (poly)peptide having at least 75% sequence identity to SEQ ID NO:6;
(vii) SNNLDSKVGGNYNYLYRLFRKSNLKPFERDISTEIYQAGSTPXNGVEGFN XYFPLQSYGFQPTNGVGYQPYRGSDIPIGAGIXASYQTQTNSPRRARSVASQSIIA YGSLLNKHIDAYKTFP (Fragment 7, SEQ ID NO:7), or an immunogenic (poly)peptide having at least 75% sequence identity to SEQ ID NO:7;
(viii) DISTEIYQAGSTPXNGVEGFNXYFPLQSYGFQPTNGVGYQPYRGSDIPIGA GIXASYQTQTNSPRRARSVASQSIIAYGSLLNKHIDAYKTFP (Fragment 8, SEQ ID NO:8), or an immunogenic (poly)peptide having at least 75% sequence identity to SEQ ID NO:8;
(ix) LLNKHIDAYKTFPGSDIPIGAGIXASYQTQTNSPRRARSVASQSIIAYGSDIS TEIYQAGSTPXNGVEGFNXYFPLQSYGFQPTNGVGYQPYR (Fragment 9, SEQ ID NO:9), or an immunogenic (poly)peptide having at least 75% sequence identity to SEQ ID NO:9;
(x) LLNKHIDAYKTFPGSDIPIGAGIXASYQTQTNSPRRARSVASQSIIAYGSSN NLDSKVGGNYNYLYRLFRKSNLKPFERDISTEIYQAGSTPXNGVEGFNXYFPLQS YGFQPTNGVGYQPYR (Fragment 10, SEQ ID NO:10), or an immunogenic (poly)peptide having at least 75% sequence identity to SEQ ID NO:10;
(xi) DIPIGAGIXASYQTQTNSPRRARSVASQSIIAYGSLLNKHIDAYKTFPGSDIS TEIYQAGSTPXNGVEGFNXYFPLQSYGFQPTNGVGYQPYR (Fragment 11, SEQ ID NO:11), or an immunogenic (poly)peptide having at least 75% sequence identity to SEQ ID NO:11;
(xii) DIPIGAGIXASYQTQTNSPRRARSVASQSIIAYGSLLNKHIDAYKTFPGSSN NLDSKVGGNYNYLYRLFRKSNLKPFERDISTEIYQAGSTPXNGVEGFNXYFPLQS YGFQPTNGVGYQPYR (Fragment 12, SEQ ID NO:12), or an immunogenic (poly)peptide having at least 75% sequence identity to SEQ ID NO:12;
(xiii) VFVSNGTHWFVTQRNFY (Fragment 13, SEQ ID NO:13), or an immunogenic (poly)peptide having at least 75% sequence identity to SEQ ID NO:13;
wherein “X” represents Cysteine, Alanine or Serine.

5. The method of claim 1, wherein the SARS-CoV2 protein, fragment or fusion product is fused to a periplasmic or lipoprotein leader sequence, which promotes the translocation of the fragment or the fragment fusion product into the periplasmic space or its insertion into the outer membrane as lipidated (poly)peptide.

6. The method of claim 1, wherein the SARS-CoV2 protein, fragment or fusion product is fused to a carrier protein which promotes its translocation into the OMV lumen or membrane.

7. The method of claim 6, wherein the SARS-CoV2 protein, fragment or fusion product is fused to the C-terminus of the Staphylococcus aureus FhuD2 protein.

8. The method of claim 1, wherein the bacterium is of the genus Escherichia, Pseudomonas, Neisseria or Shigella.

9. The method of claim 8, wherein the bacterium is E. coli.

10. the method of claim 1, wherein the bacterium carries gene-inactivating mutations at the ompA gene and at one or more of the following genes which encode proteins naturally present in the OMVs: ybis, ais, eco, glpQ, mltA, proX, ydcL, glnH, efeO, bglX, agp, ygdI, yncD, slp, artI, yiaD, ompX, borD, yhiJ, emtA, fecA, nmpC, fhuA, hisJ, lamB, malE, malM, ygiW, cirA, fepA, loiP, yjeI, ecnB, rcsF, phoE, oppA, fkpA, ybaY, tsx, yggE, osmE, ygdR, yceI, bhsA, nlpE, pldA, yghJ, ydeN, ushA, mdoD, treA, bcsC, ftsP, ptrA, fadL, artJ, mlaA,

11. The method of claim 10, wherein the bacterium carries further gene-inactivating mutations at the msbB and pagP genes.

12. The method of claim 1, wherein the OMVs are separated from the culture medium by filtration.

13. The method of claim 1, wherein the OMVs are purified by centrifugation or ultrafiltration.

14. (canceled)

15. Isolated outer membrane vesicles (OMVs) carrying a SARS-CoV2 protein or fragment or fusion product as defined in claim 1, in the lumen or in the membrane.

16. (canceled)

17. An immunogenic composition comprising outer membrane vesicles according to claim 15, optionally in combination with pharmaceutically acceptable adjuvants and excipients.

18. The immunogenic composition of claim 17, which is in the form of a vaccine suitable for oral, parenteral, nasal, ocular, transmucosal, intradermal or intramuscular administration.

19. A method of stimulating an immune response in a subject in need thereof with an outer membrane vesicles according to claim 15, optionally in combination with pharmaceutical acceptable adjuvants and excipients, said method comprising
administering a pharmaceutical acceptable amount of said outer membrane vesicles to said subject in need thereof, said outer membrane vesicles being optionally in combination with pharmaceutically acceptable adjuvants and excipients.

20. The method according to claim 19, wherein said stimulation of an immune response in said subject is applied to prophylaxis or treatment of SARS-CoV2 infection.


SEQUENCE LISTING (41 sequences)

SEQ ID NO: 1 - protein, 254 aa, Coronavirus
  X (7)..(7): any natural amino acid
  X (32)..(32): any natural amino acid
  X (50)..(50): any natural amino acid
  X (62)..(62): any natural amino acid
  X (103)..(103): any natural amino acid
  X (196)..(196): any natural amino acid
  X (209)..(209): any natural amino acid
  X (209)..(209): any natural amino acid
PNITNLXPFGEVFNATRFASVYAWNRKRISNXVADYSVLYNSASFSTFKXYGVSPTKLNDLXFTNVYADSFVIRGDEVRQIAPGQTGKIADYNYKLPDDFTGXVIAWNSNNLDSKVGGNYNYLYRLFRKSNLKPFERDISTEIYQAGSTPCNGVEGFNCYFPLQSYGFQPTNGVGYQPYRVVVLSFELLHAPATVXGPKKSTNLVKNKXVNFNFNGLTGTGVLTESNKKFLPFQQFGRDIADTTDAVRDPQTLE

SEQ ID NO: 2 - protein, 72 aa, Coronavirus
SNNLDSKVGGNYNYLYRLFRKSNLKPFERDISTEIYQAGSTPCNGVEGFNCYFPLQSYGFQPTNGVGYQPYR

SEQ ID NO: 3 - protein, 25 aa, Coronavirus
SNNLDSKVGGNYNYLYRLFRKSNLK

SEQ ID NO: 4 - protein, 43 aa, Coronavirus
DISTEIYQAGSTPCNGVEGFNCYFPLQSYGFQPTNGVGYQPYR

SEQ ID NO: 5 - protein, 33 aa, Coronavirus
  X (9)..(9): any natural amino acid
DIPIGAGIXASYQTQTNSPRRARSVASQSIIAY

SEQ ID NO: 6 - protein, 13 aa, Coronavirus
LLNKHIDAYKTFP

SEQ ID NO: 7 - protein, 122 aa, Coronavirus
  X (43)..(43): any natural amino acid
  X (51)..(51): any natural amino acid
  X (83)..(83): any natural amino acid
SNNLDSKVGGNYNYLYRLFRKSNLKPFERDISTEIYQAGSTPXNGVEGFNXYFPLQSYGFQPTNGVGYQPYRGSDIPIGAGIXASYQTQTNSPRRARSVASQSIIAYGSLLNKHIDAYKTFP

SEQ ID NO: 8 - protein, 93 aa, Coronavirus
  X (14)..(14): any natural amino acid
  X (22)..(22): any natural amino acid
  X (54)..(54): any natural amino acid
DISTEIYQAGSTPXNGVEGFNXYFPLQSYGFQPTNGVGYQPYRGSDIPIGAGIXASYQTQTNSPRRARSVASQSIIAYGSLLNKHIDAYKTFP

SEQ ID NO: 9 - protein, 93 aa, Coronavirus
  X (24)..(24): any natural amino acid
  X (64)..(64): any natural amino acid
  X (72)..(72): any natural amino acid
LLNKHIDAYKTFPGSDIPIGAGIXASYQTQTNSPRRARSVASQSIIAYGSDISTEIYQAGSTPXNGVEGFNXYFPLQSYGFQPTNGVGYQPYR

SEQ ID NO: 10 - protein, 122 aa, Coronavirus
  X (24)..(24): any natural amino acid
  X (93)..(93): any natural amino acid
  X (101)..(101): any natural amino acid
LLNKHIDAYKTFPGSDIPIGAGIXASYQTQTNSPRRARSVASQSIIAYGSSNNLDSKVGGNYNYLYRLFRKSNLKPFERDISTEIYQAGSTPXNGVEGFNXYFPLQSYGFQPTNGVGYQPYR

SEQ ID NO: 11 - protein, 93 aa, Coronavirus
  X (9)..(9): any natural amino acid
  X (64)..(64): any natural amino acid
  X (72)..(72): any natural amino acid
DIPIGAGIXASYQTQTNSPRRARSVASQSIIAYGSLLNKHIDAYKTFPGSDISTEIYQAGSTPXNGVEGFNXYFPLQSYGFQPTNGVGYQPYR

SEQ ID NO: 12 - protein, 122 aa, Coronavirus
  X (9)..(9): any natural amino acid
  X (93)..(93): any natural amino acid
  X (101)..(101): any natural amino acid
DIPIGAGIXASYQTQTNSPRRARSVASQSIIAYGSLLNKHIDAYKTFPGSSNNLDSKVGGNYNYLYRLFRKSNLKPFERDISTEIYQAGSTPXNGVEGFNXYFPLQSYGFQPTNGVGYQPYR

SEQ ID NO: 13 - protein, 17 aa, Coronavirus
VFVSNGTHWFVTQRNFY

SEQ ID NO: 14 - DNA, 25 bp, Artificial Sequence
  oligonucleotide primer
catcaccatcaccatcacgattaca

SEQ ID NO: 15 - DNA, 23 bp, Artificial Sequence
  oligonucleotide primer
gctggagcaacctgccagcagag

SEQ ID NO: 16 - DNA, 21 bp, Artificial Sequence
  oligonucleotide primer
gccctggaagtacaggttttc

SEQ ID NO: 17 - DNA, 22 bp, Artificial Sequence
  oligonucleotide primer
cgcgacttaattctagcataac

SEQ ID NO: 18 - DNA, 57 bp, Artificial Sequence
  oligonucleotide primer
gatattccaatcggcgcaggtatttgtgcatcttaccagacccagaccaatagccca

SEQ ID NO: 19 - DNA, 57 bp, Artificial Sequence
  oligonucleotide primer
atacgcaataatgctttgggaagccacggaacgagcacgacgtgggctattggtctg

SEQ ID NO: 20 - DNA, 65 bp, Artificial Sequence
  oligonucleotide primer
agcattattgcgtatggcagcgatatcccgattggtgcgggtatttgcgcaagctaccagacgca

SEQ ID NO: 21 - DNA, 70 bp, Artificial Sequence
  oligonucleotide primer
gtacgcgataatagactgagatgcgacagagcgagcacgacgcggagagttggtctgcgtctggtagctt

SEQ ID NO: 22 - DNA, 43 bp, Artificial Sequence
  oligonucleotide primer
taattaaagctgcaaaagatattagcaccgaaatttatcaggc

SEQ ID NO: 23 - DNA, 40 bp, Artificial Sequence
  oligonucleotide primer
gatggtgatggtgatgttaacgatacggctgataacccac

SEQ ID NO: 24 - DNA, 37 bp, Artificial Sequence
  oligonucleotide primer
ccgtttgaacgtgatattagcaccgaaatttatcagg

SEQ ID NO: 25 - DNA, 39 bp, Artificial Sequence
  oligonucleotide primer
taattaaagctgcaaaaagcaataacctggatagcaaag

SEQ ID NO: 26 - DNA, 34 bp, Artificial Sequence
  oligonucleotide primer
acgttcaaacggtttcagattgctcttacgaaac

SEQ ID NO: 27 - DNA, 38 bp, Artificial Sequence
  oligonucleotide primer
ctgtacttccagggcccgaatatcaccaatctgtgtcc

SEQ ID NO: 28 - DNA, 41 bp, Artificial Sequence
  oligonucleotide primer
tagaattaagtcgcgttattattccagtgtctgcggatcac

SEQ ID NO: 29 - DNA, 39 bp, Artificial Sequence
  oligonucleotide primer
taattaaagctgcaaaagatattccaatcggcgcaggta

SEQ ID NO: 30 - DNA, 41 bp, Artificial Sequence
  oligonucleotide primer
gatggtgatggtgatgttagtacgcgataatagactgagat

SEQ ID NO: 31 - DNA, 765 bp, Coronavirus
ccgaatatcaccaatctgtgtccgtttggtgaagtttttaatgcaacccgttttgcaagcgtttatgcctggaatcgtaaacgtattagcaattgcgttgccgattatagcgttctgtataatagcgcaagcttcagcacctttaaatgctatggtgttagcccgaccaaactgaatgatctgtgttttaccaatgtgtatgccgatagctttgtgattcgtggtgatgaagttcgtcagattgcaccgggtcagaccggtaaaattgcagattataactataaactgccggatgattttacgggttgtgttattgcatggaatagcaataacctggatagcaaagttggtggcaactataactatctgtatcgcctgtttcgtaagagcaatctgaaaccgtttgaacgtgatattagcaccgaaatttatcaggcaggtagcaccccgtgcaatggtgttgaaggttttaattgttattttccgctgcagagctatggttttcagccgacaaatggtgtgggttatcagccgtatcgtgttgttgttctgtcatttgaactgctgcatgcaccggcaaccgtttgtggtccgaaaaaaagtaccaatctggtgaaaaacaagtgcgtgaactttaactttaatggtctgaccggcaccggtgttctgaccgaaagcaataaaaagtttctgccgtttcagcagtttggccgtgatattgcagataccaccgatgcagttcgtgatccgcagacactggaataa

SEQ ID NO: 32 - protein, 254 aa, Coronavirus
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 33 - DNA, 1047 bp, Artificial Sequence
  plasmidic DNA encoding FhuD2-Fragment 4
atgatgaaagctactaaactggtactgggcgcggtaatcctgggttctactctgctggcaggttgcgggaaccaaggtgaaaaaaataacaaagctgaaactaaatcttataaaatggacgatggcaaaacggtagatattccgaaagaccctaaacgcattgcagtagttgcgccaacatatgctggtggacttaaaaaattaggtgcaaacattgtagctgtaaatcaacaagtcgatcaaagcaaagtattaaaagataaatttaaaggtgttacaaaaattggtgatggcgatgtagaaaaagttgctaaagaaaagccagatttaattattgtatactctactgacaaagatattaaaaaatatcaaaaagtagcaccaacagtagttgttgactataataagcataaatatttagaacaacaagaaatgttagggaaaattgttggtaaagaagataaagtaaaagcttggaagaaagattgggaagaaacaactgctaaagacggtaaagaaattaaaaaagcaattggacaagatgcaacagtgtcattgtttgatgaatttgataaaaaattatacacttacggcgataactggggtcgtggtggagaagtattatatcaagcatttggtttgaaaatgcaaccagaacaacaaaagttaactgcaaaagcaggttgggctgaagtgaaacaagaagaaattgaaaaatatgctggtgattacattgtgagtacaagtgaaggtaaacctacaccaggatacgaatcaacaaacatgtggaagaatttgaaagctactaaagaaggacatattgttaaagttgatgctggtacatactggtacaacgatccttatacattagatttcatgcgtaaagatttaaaagaaaaattaattaaagctgcaaaagatattagcaccgaaatttatcaggcaggtagcaccccgtgcaatggtgttgaaggttttaattgttattttccgctgcagagctatggttttcagccgacaaatggtgtgggttatcagccgtatcgt

SEQ ID NO: 34 - protein, 349 aa, Artificial Sequence
  FhuD2-Fragment 4
MMKATKLVLGAVILGSTLLAGCGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAKDISTEIYQAGSTPCNGVEGFNCYFPLQSYGFQPTNGVGYQPYR

SEQ ID NO: 35 - DNA, 1134 bp, Artificial Sequence
  plasmidic DNA encoding FhuD2-Fragment 2
atgatgaaagctactaaactggtactgggcgcggtaatcctgggttctactctgctggcaggttgcgggaaccaaggtgaaaaaaataacaaagctgaaactaaatcttataaaatggacgatggcaaaacggtagatattccgaaagaccctaaacgcattgcagtagttgcgccaacatatgctggtggacttaaaaaattaggtgcaaacattgtagctgtaaatcaacaagtcgatcaaagcaaagtattaaaagataaatttaaaggtgttacaaaaattggtgatggcgatgtagaaaaagttgctaaagaaaagccagatttaattattgtatactctactgacaaagatattaaaaaatatcaaaaagtagcaccaacagtagttgttgactataataagcataaatatttagaacaacaagaaatgttagggaaaattgttggtaaagaagataaagtaaaagcttggaagaaagattgggaagaaacaactgctaaagacggtaaagaaattaaaaaagcaattggacaagatgcaacagtgtcattgtttgatgaatttgataaaaaattatacacttacggcgataactggggtcgtggtggagaagtattatatcaagcatttggtttgaaaatgcaaccagaacaacaaaagttaactgcaaaagcaggttgggctgaagtgaaacaagaagaaattgaaaaatatgctggtgattacattgtgagtacaagtgaaggtaaacctacaccaggatacgaatcaacaaacatgtggaagaatttgaaagctactaaagaaggacatattgttaaagttgatgctggtacatactggtacaacgatccttatacattagatttcatgcgtaaagatttaaaagaaaaattaattaaagctgcaaaaagcaataacctggatagcaaagttggtggcaactataactatctgtatcgcctgtttcgtaagagcaatctgaaaccgtttgaacgtgatattagcaccgaaatttatcaggcaggtagcaccccgtgcaatggtgttgaaggttttaattgttattttccgctgcagagctatggttttcagccgacaaatggtgtgggttatcagccgtatcgt

SEQ ID NO: 36 - protein, 378 aa, Artificial Sequence
  FhuD2-Fragment 2
MMKATKLVLGAVILGSTLLAGCGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAKSNNLDSKVGGNYNYLYRLFRKSNLKPFERDISTEIYQAGSTPCNGVEGFNCYFPLQSYGFQPTNGVGYQPYR

SEQ ID NO: 37 - DNA, 1122 bp, Artificial Sequence
  plasmidic DNA encoding FhuD2-Fragment 5
atgatgaaagctactaaactggtactgggcgcggtaatcctgggttctactctgctggcaggttgcgggaaccaaggtgaaaaaaataacaaagctgaaactaaatcttataaaatggacgatggcaaaacggtagatattccgaaagaccctaaacgcattgcagtagttgcgccaacatatgctggtggacttaaaaaattaggtgcaaacattgtagctgtaaatcaacaagtcgatcaaagcaaagtattaaaagataaatttaaaggtgttacaaaaattggtgatggcgatgtagaaaaagttgctaaagaaaagccagatttaattattgtatactctactgacaaagatattaaaaaatatcaaaaagtagcaccaacagtagttgttgactataataagcataaatatttagaacaacaagaaatgttagggaaaattgttggtaaagaagataaagtaaaagcttggaagaaagattgggaagaaacaactgctaaagacggtaaagaaattaaaaaagcaattggacaagatgcaacagtgtcattgtttgatgaatttgataaaaaattatacacttacggcgataactggggtcgtggtggagaagtattatatcaagcatttggtttgaaaatgcaaccagaacaacaaaagttaactgcaaaagcaggttgggctgaagtgaaacaagaagaaattgaaaaatatgctggtgattacattgtgagtacaagtgaaggtaaacctacaccaggatacgaatcaacaaacatgtggaagaatttgaaagctactaaagaaggacatattgttaaagttgatgctggtacatactggtacaacgatccttatacattagatttcatgcgtaaagatttaaaagaaaaattaattaaagctgcaaaagatattccaatcggcgcaggtatttgtgcatcttaccagacccagaccaatagcccacgtcgtgctcgttccgtggcttcccaaagcattattgcgtatggcagcgatatcccgattggtgcgggtatttgcgcaagctaccagacgcagaccaactctccgcgtcgtgctcgctctgtcgcatctcagtctattatcgcgtac

SEQ ID NO: 38 - protein, 374 aa, Artificial Sequence
  FhuD2-Fragment 5
MMKATKLVLGAVILGSTLLAGCGNQGEKNNKAETKSYKMDDGKTVDIPKDPKRIAVVAPTYAGGLKKLGANIVAVNQQVDQSKVLKDKFKGVTKIGDGDVEKVAKEKPDLIIVYSTDKDIKKYQKVAPTVVVDYNKHKYLEQQEMLGKIVGKEDKVKAWKKDWEETTAKDGKEIKKAIGQDATVSLFDEFDKKLYTYGDNWGRGGEVLYQAFGLKMQPEQQKLTAKAGWAEVKQEEIEKYAGDYIVSTSEGKPTPGYESTNMWKNLKATKEGHIVKVDAGTYWYNDPYTLDFMRKDLKEKLIKAAKDIPIGAGICASYQTQTNSPRRARSVASQSIIAYGSDIPIGAGICASYQTQTNSPRRARSVASQSIIAY

SEQ ID NO: 39 - DNA, 132 bp, Coronavirus
gatattagcaccgaaatttatcaggcaggtagcaccccgtgcaatggtgttgaaggttttaattgttattttccgctgcagagctatggttttcagccgacaaatggtgtgggttatcagccgtatcgttaa

SEQ ID NO: 40 - DNA, 219 bp, Coronavirus
agcaataacctggatagcaaagttggtggcaactataactatctgtatcgcctgtttcgtaagagcaatctgaaaccgtttgaacgtgatattagcaccgaaatttatcaggcaggtagcaccccgtgcaatggtgttgaaggttttaattgttattttccgctgcagagctatggttttcagccgacaaatggtgtgggttatcagccgtatcgttaa

SEQ ID NO: 41 - DNA, 257 bp, Coronavirus
gatattccaatcggcgcaggtatttgtgcatcttaccagacccagaccaatagcccacgtcgtgctcgttccgtggcttcccaaagcattattgcgtatggcagcgatatcccgattggtgcgggtatttgcgcaagctaccagacgcaggcagcgatatcccgattggtgcgggtatttgcgcaagctaccagacgcagaccaactctccgcgtcgtgctcgctctgtcgcatctcagtctattatcgcgtactaa